(19) United States
(12) Patent Application Publication
(10) Pub. No.: US 2018/0206834 A1
(43) Pub. Date: Jul. 26, 2018

(54) System for Approaching the Spine Laterally and Retracting Tissue in an Anterior to Posterior Direction

(71) Applicant: MIS IP Holdings, LLC, Denver, CO (US)

(72) Inventors: Fernando Villamil, San Juan, PR (US); Brandon Arthurs, Wilmington, NC (US); Ryan Arce, Denver, CO (US); Jeffrey Schell, Denver, CO (US); Leighton LaPierre, Thornton, CO (US)

(73) Assignee: MIS IP Holdings, LLC, Denver, CO (US)

(21) Appl. No.: 15/862,257

(22) Filed: Jan. 4, 2018

Related U.S. Application Data
(60) Provisional application No. 62/442,356, filed on Jan. 4, 2017.

(51) Int. Cl. A61B 17/02

(52) U.S. Cl. CPC A61B 17/025; A61F 2/4611; A61B 17/0218; A61B 17/0206

(57) ABSTRACT

The invention now summarized here is directed toward a surgical device that enables utilization of the lateral approach to the spine with the ability to retract tissue in an anterior-to-posterior direction. The invention also incorporates a surgical method for utilization of the above-mentioned surgical device, embodiments of which incorporate steps for approaching the anterior portion of the disc space, retracting soft tissue in a generally posterior direction, removing disc material, placing a bone graft and removing the associated instrumentation.

### CROSS REFERENCE TO RELATED APPLICATIONS

This application claims benefit to U.S. Provisional Patent Application No. 62/442,356, entitled “System for Approaching the Spine Laterally and Anterior to the Psoas Muscle” filed Jan. 4, 2017, which is incorporated by reference in its entirety for all purposes.

### BACKGROUND OF THE INVENTION

A number of approaches, systems and apparatuses have been devised to accomplish a variety of surgical interventions in association with the spine. These approaches enable a surgeon to place instrumentation and implantable apparatuses related to discectomy, laminectomy, spinal fusion, vertebral body replacement and other procedures intended to address pathologies of the spine. The variety of surgical approaches to the spine have a number of advantages and drawbacks such that no one perfect approach exists. A surgeon often chooses one surgical approach to the spine from a multitude of options dependent on the relevant anatomy, pathology and a comparison of the advantages and drawbacks of the variety of approaches relevant to a particular patient.

A common approach to the spine, that has increased in popularity for use especially in association with spinal fusion, is the lateral approach. The lateral approach used in association with spinal fusion is more commonly referred to as lateral lumbar interbody fusion (or “LLIF”). Variants of this approach are also commonly referred to as the “direct lateral” approach in association with the “DLIF” procedure and the “extreme lateral” approach in association with the “XLIF” procedure. Lateral approaches, in general, require a surgeon to access the spine by creating a path through the side of the patient's body through the psoas muscle.

Lateral approaches have a variety of advantages over other approaches. For instance, unlike an anterior approach commonly utilized in association with anterior lumbar interbody fusion (or “ALIF”), the lateral approach generally avoids the need for a surgeon to interact with the great vessels, such as the vena cava and the aorta, an inadvertent puncture of which could cause death to the patient. A lateral approach also allows a surgeon to avoid the need to remove the facet joint, an important supportive structure of the spine, to place implants as generally takes place during the trans-foraminal approach during trans-foraminal lumbar interbody fusion (or “TLIF”). The lateral approach also allows for the placement of a relatively large interbody implant compared especially to a TLIF procedure, enabling the incorporation of more bone graft and a more dispersive distribution of the weight of the spine through the implant, and contact between the implant and the epiphyseal ring of the vertebral bodies, thereby minimizing risk of subsidence of the implant. The lateral approach also is well-suited to enable a surgeon to place a lordotic implant pre-formed to help a surgeon ensure a desirable curvature to the spine after spinal fusion is accomplished.

To aid in describing the various locations and trajectories of involving the interbody disc and disc space, in surgical interventions involving the interbody disc space of the spine including interventions utilizing the lateral approach to the spine, the disc and enclosing area may be nominally divided into four quadrants to help describe and identify the target of the approach trajectory. Such quadrants may be numerically labeled in an ascending manner from “1” to “4,” anterior to posterior in reference to the patient's body and proximal to distal from the surgeon's location. Therefore, the labelling may be as follows: anterior-proximal quadrant is labelled “1,” the anterior-distal quadrant is labelled “2,” the posterior-proximal quadrant is labelled “3,” and the posterior-distal quadrant is labelled “4.” (Moro et al. 2003)

A major problem associated with the lateral surgical approach to the spine is nerve damage. The lumbar plexus is a web of nerves (a nervous plexus) in the lumbar region of the body which forms part of the larger lumbosacral plexus. It is formed by the divisions of the first four lumbar nerves (L1-L4) and from contributions of the subcostal nerve (T12), which is the last thoracic nerve. The lumbar plexus in particular is often damaged as a direct result of surgical intervention utilizing the lateral approach to the spine. The nerves associated with the lumbar plexus can experience indirect nerve injury as a result of over-dilation or over-retraction of apparatuses utilized to accomplish lateral access to the spine. They also can experience direct nerve injury as a result of direct trauma caused by interaction from the instrumentation utilized during the surgical intervention in association with the lateral approach to the spine.

Over-retraction commonly occurs in surgeries utilizing the direct lateral and extreme lateral approaches when a retractor instrument used during the procedure is placed too close to a nerve structure, such as the lumbar plexus. The nerves close to the spine generally orient in a cephalad-caudal trajectory substantially parallel to the axis of the spine. However, surgical interventions utilizing the direct lateral and extreme lateral approaches generally require the retraction or redirection of the nerves in the anterior-posterior plane. As a result, this retraction causes a stretching, or elongation of the nerve, which damages the nerve. This nerve trauma resulting from over-retraction, especially in relation to the lumbar plexus, manifests in a variety of undesirable consequences to a patient post-surgery. These undesirable consequences include dystesthia, numbness, burning and tingling in the leg, especially in the anterior thigh. Moreover, a patient who suffers from nerve trauma during a surgical intervention utilizing the lateral approach also may experience palsy or muscle weakness. The patient may also experience problems associated with genitalia, including retrograde ejaculation, impotence and incontinence as a direct result of the nerve injury during by surgical intervention utilizing the direct lateral or extreme lateral approaches. It follows that a need remains to create an improved approach to the spine and that an improved technique is therefore desirable to avoid the risk of such post-surgical complications to patients.

In addition to the more commonly experienced indirect nerve injuries associated with surgical interventions utilizing the lateral approach such as those as described in the preceding paragraph, such interventions are also accompanied by a risk, though less prevalent, of direct nerve injuries. For instance, the currently known systems and apparatuses associated with surgical interventions utilizing the lateral approach risk directly tearing or lacerating nerve structures. The trajectory undertaken by surgeons through the muscle is the main culprit of such risk, as the trajectory requires the surgeon to traverse near to and sometimes in contact with nerve structures. The design of bladed instrumentation also allows for nerve injury in many cases, as blunt-edged blades known in the prior art can lacerate or tear nerves when such blades come into contact with nerve structures. An improved surgical approach and associated improved instruments to accomplish surgical intervention with less risk of direct nerve injury are therefore desirable.

A major problem associated with surgical interventions utilizing the lateral approach to the spine is that they require some type of nerve mapping that utilizes neuro-monitoring techniques, including Electromyography (“EMG”). Such neuro-monitoring techniques assist surgeons to identify the locations of nerves and to avoid causing damage to the nerves during the surgical approach. Typical neuro-monitoring techniques, such as free run EMG or triggered EMG (also known as tEMG), however, cannot detect all types of nerves. Only motor nerves, and not sensory nerves, can be detected by standard neuro-monitoring techniques, such as EMG and tEMG, which are typically used in association with surgical interventions utilizing the lateral approach to accomplish spinal fusion.

Relatedly, the nerve structures have influenced the development of the lateral technique by spine surgeons. As most motor nerves of the spine tend to occur in or near zones 3 and 4 of the interbody space (the posterior portion) (Moro et al. 2003), and most sensory nerves tend to occur in or near zones 1 and 2 (the anterior portion) (Banagan et al. Spine, 2011), most lateral approaches to the spine target zones 3 and 4 as neuro-monitoring can detect the motor nerves in that area (Malham et al. 2012). As sensory nerves, including and especially the genitofemoral nerve (GFN), occur in zones 1 and 2 of the interbody space (the anterior portion), most lateral techniques are specifically designed to avoid zones 1 and 2 as the neuro-monitoring techniques typically used in association with surgical interventions utilizing the lateral approach cannot detect such sensory nerves.

Many problems are associated with surgical interventions utilizing the lateral approaches that target zones 3 and 4 because of their targeting of the posterior area of the disc space. First, the motor nerves that exist in and/or near the posterior portion of the disc space are generally larger than the sensory nerves. Therefore, it is more likely that a retractor utilized in association with the lateral approach would come into direct contact with the nerves. As the motor nerves are larger and thereby less elastic and less pliable, the motor nerves have a greater likelihood of indirect damage especially resulting from the elongation or stretching of the nerves related to over-retraction or extended time of retraction. (Davis et al. Bone Joint Surg Am, 2011) Therefore, a need exists for an alternative approach that avoids targeting zones 3 and 4 of the spine to avoid direct and indirect damage to the motor nerves, while mitigating damage to the sensory nerves that cannot be detected by neuro-monitoring techniques typically utilized in association with surgical techniques utilizing the lateral approach (Banagan et al 2011).

In addition to the risk of nerve damage, a significant risk of damage to the musculature surrounding the spine and associated complications accompanies the use of the lateral approach in surgical interventions associated with the spine. In typical lateral approaches, after making an incision, the surgeon will place a number of sequential dilators on the desired pathway to the spine through the psoas muscle, and retract the psoas muscle and other soft tissues through use of a bladed retractor apparatus. However, a common problem associated with this type of lateral procedure is that soft tissues, including musculature and nerves become trapped near the distal end of the retractor's blades (often referred to as “trappage”). An associated problem is the time and effort it takes for a surgeon to utilize a cautery or similar device to remove the trapped soft tissues from between the distal end of the retractor and the vertebral bodies prior to completing access to the spine.

Often, the resulting damage and trauma to the soft tissue resulting from trappage and removal of psoas muscle tissue with a cautery causes lasting problems for a patient. For instance, a patient who experiences trappage during surgery will often have lower body pain and leg weakness. Such pain and leg weakness occurs due to the linkage of the psoas to the lower body, as the psoas muscle connects to the femur. Thus, damage to the psoas will generally manifest in lower body discomfort, including pain and weakness in the leg. Generally, the psoas muscle is larger near the posterior portion of the disc space than near the anterior portion of the disc space.

Nerve and muscle damage during the lateral approach is a heavily documented problem. For instance, transient post-operative motor palsy has been reported in up to 25% of standard LLIF procedures and permanent sensory dysesthesia in up to 63% of standard LLIF procedures (Youssef et al. Spine 2010). These significant and troublesome complication rates are directly associated with nerve and muscle damage. Therefore, a need remains for an improved lateral approach to the spine, utilizing improved apparatuses and techniques, which harnesses the realized advantages of the lateral approach while minimizing the drawbacks and complications associated with surgical procedures utilizing the lateral approach.

While avoiding the bulk of the psoas muscle during the lateral surgical approach would mitigate many of the drawbacks to the lateral approach, other trajectories pose alternative risks. For instance, specifically moving the lateral surgical approach trajectory anterior to the psoas muscle or through the anterior portion of the psoas muscle risks other consequences. Specifically, an approach that targets the anterior third of the disc space would increase the risk of damage to the vena cava and aorta, also known as the “great vessels.” As the great vessels lie generally proximal and anterior to the spine, any approach targeting the anterior anatomy of the spine would increase the risk of damaging the great vessels. A puncture of the great vessels during surgery would cause bleeding out of the vessels at a high rate and could lead to death. Spine surgeons therefore are often hesitant to utilize techniques that traverse near the anterior of a spine without the assistance or support of a vascular surgeon who can potentially help the spine surgeon avoid the vascular structures or assist in the emergency repair of a vascular structure damaged during the surgical approach.

A separate but related problem associated with changing the trajectory of the lateral approach to target the anterior third of the disc space relates to the current constraints of the surgical instrumentation used during surgery associated with the lateral approach, including especially the retractors. The present retractors utilized in association with the lateral approach to the spine are designed to move the soft tissues surrounding the spine in a specific trajectory. This derives in part from the primacy of lateral approach techniques that target zones 3 and 4 (associated with the posterior portion of the disc space) while avoiding zones 1 and 2 (associated with the anterior portion of the disc space). Specifically, the known retractors used in association with lateral approaches target zones 3 and 4. These prior art retractors expand such that the retractor components push the nerve structures and musculature in or near zones 3 and 4 in an anterior direction into or near zones 1 and 2. In order to accomplish a surgical intervention utilizing the lateral approach anterior to the psoas muscle or through the anterior portion of the psoas muscle, retractors known in the prior art are generally not useful, as they are configured to push the soft tissues in and near the spine in an anterior direction. A need therefore remains for a soft tissue retraction device that in contrast pushes soft tissues, including the nerve structures (such as the genitofemoral nerve, also known as the “GFN”) and the musculature, in or near zones 1 and 2 in a generally posterior direction into or near zones 3 and 4, while mitigating the unique risks of damage to soft tissues posed by pushing in a generally anterior direction.

A problem related to the movement of the soft tissues from areas in or near the anterior portion of the disc space to areas in or near the posterior portion of the disc space relates to the risk of elongating the GFN, causing nerve trauma. It remains to be discovered how a retractor system intended to relocate the soft tissues near zones 1 and 2 in a generally posterior direction can avoiding elongation of the genitofemoral nerve or GFN. While, generally, there are advantageously less musculature and nerve structures in and around zones 1 and 2, the genitofemoral nerve (undetectable by neuro-monitoring techniques typically used in association with lateral approaches) still generally resides in and near zone 1 and 2 of the interbody space (Banagan et al. 2011). The GFN, as a smaller sensory nerve, though undetectable by neuro-monitoring techniques typically utilized in association with the lateral approach, is more pliable than the larger motor nerves. Therefore, they are subject to less risk of traumatic elongation due to over-retraction of a retractor utilized in association with the lateral approach than the larger motor nerves.

### BRIEF SUMMARY OF THE INVENTION

Conventional techniques in spinal fusion utilizing the lateral approach to the spine typically retract tissue in a posterior-to-anterior direction. The invention now summarized here is directed toward a surgical device that enables utilization of the lateral approach to the spine with the ability to retract tissue in an anterior-to-posterior direction. Embodiments of the inventive tissue retraction system require less manipulation of the psoas muscle and less risk of damage to the motor nerves during surgery than do currently available spinal retractor systems, thus placing collateral soft tissue at a lesser risk of damage, and generally improving the efficiency and safety of the surgical procedure.

The invention provides a retractor system for facilitating spinal surgery and methods of surgery that use the system. The retractor system includes a multi-bladed retractor apparatus capable of shielding the operating channel from the at risk structures slightly anterior to the operating channel, thus minimizing the risks of approaching the anterior aspects of the disc space.

Some embodiments of the retractor system are configured to accommodate an oval dilation system. In some embodiments, the oval dilation system is configured to minimize damage to the psoas muscle by aligning the length of the retractor in a plane parallel to the direction of the muscle fibers and thereby minimize cutting of the muscle fibers.

The invention, as recited above, also provides a method for spinal surgery that makes use of the above-summarized system. Embodiments of the method include approaching the anterior aspect of the disc space through use of tools associated with the above-summarized system, expanding the operating channel, approaching the disc space with discectomy tools, placing an interbody graft and then removing the instrumentation.

### BRIEF DESCRIPTION OF THE DRAWINGS

FIG. 1A: a perspective view of an embodiment of a dilator 1400;

FIG. 1B: a lateral view of an embodiment of a dilator 1400;

FIG. 2A: a perspective view of an embodiment of an elongate member 1410 enclosed within an embodiment of a dilator 1400;

FIG. 2B: a lateral view of an embodiment of an elongate member 1410 enclosed within a an embodiment of a dilator 1400;

FIG. 3A a perspective view of an embodiment of an elongate member 1410 enclosed within an embodiment of a dilator 1400 enclosed within an embodiment of a final dilator 1420;

FIG. 3B: a lateral view of an embodiment of an elongate member 1410 enclosed within an embodiment of a dilator 1400 enclosed within an embodiment of a final dilator 1420;

FIG. 4: a perspective view of an embodiment of the multi-bladed retractor assembly 1000, with an embodiment of the retractor blades 1100 in closed position enclosing an embodiment of a final dilator 1420;

FIG. 5A: A perspective view of a portion of an embodiment of the multi-bladed retractor assembly 1000 incorporating an embodiment of the safety barrier shim 1160 positioned in the anterior aspect of the disc space;

FIG. 5B: A lateral view of an embodiment of the multi-bladed retractor assembly 1000 enclosing an embodiment of a final dilator 1420;

FIG. 6: A perspective view of an embodiment of the multi-bladed retractor assembly 1000 in the closed position without dilators;

FIG. 7: A perpendicular view of an embodiment of the stationary retractor blade 1110 incorporating an embodiment of the safety barrier shim 1160 positioned in the anterior aspect of the disc space;

FIG. 8A: A perspective view of an embodiment of the multi-bladed retractor assembly 1000 in the open position without dilators;

FIG. 8B: A lateral view of an embodiment of the multi-bladed retractor assembly 1000 in the open position without dilators;

FIG. 9A: A perspective view of an embodiment of the multi-bladed retractor assembly 1000 in the open position without dilators;

FIG. 9B: A lateral view of an embodiment of the multi-bladed retractor assembly 1000 in the open position without dilators;

FIG. 10A: A lateral view of the oval shaped dilator 1430 at initial placement in an embodiment;

FIG. 10B: A perspective view of the oval shaped dilator 1430 at initial placement in an embodiment;

FIG. 11: A lateral view of the oval shaped dilator 1430 following the rotation step in an embodiment;

FIG. 12A: A lateral view of an embodiment of the multi-bladed retractor apparatus 1000 placed over an embodiment of the oval shaped dilator 1430;

FIG. 12B: A perspective view of an embodiment of the multi-bladed retractor apparatus 1000 placed over an embodiment of the oval shaped dilator 1430;

FIG. 13A: A lateral view of the sensory nerve 1300.

FIG. 13B: A perspective view of an embodiment of the multi-bladed retractor assembly 1000 in relation to the sensory nerve 1300;

FIG. 14A: A view of an embodiment of a portion of the quick connect receptacle 1520.

FIG. 14B: A view of an embodiment of a portion of the quick connect compressive mechanism 1530 interacting with an embodiment of the quick connect receptacle 1520.

FIG. 15A: A top-down view of an embodiment of the quick connect receptacle 1520 of an embodiment of a retractor arm coupled with a retractor blade 1100.

FIG. 15B. A perspective view of an embodiment of a retractor blade 1100 incorporating an embodiment of a male retractor blade connect protrusion 1510.

FIG. 16. A lateral view of an embodiment of the multi-bladed retractor assembly 1000 in the open position following toeing of a distal retractor blade 1130.

FIG. 17. A perspective view of an embodiment of the multi-bladed retractor assembly 1000 featuring a toeing actuator 1060 and a toeing hinge 1040.

FIG. 18. A top-down view of an embodiment of a retractor arm.

FIG. 19. A perspective view of an embodiment of an oval shaped dilator 1430.

FIG. 20. A perspective view of an embodiment of an oval dilation retractor 1005.

FIG. 21A. A top-down view of an embodiment of an oval dilation retractor 1005.

FIG. 21B. A close in top-down view of an embodiment of an oval dilation retractor 1005.

FIG. 21C. A perspective view of an embodiment of an oval dilation retractor 1005 in closed position.

FIG. 22A. A top-down view of an embodiment of an oval dilation retractor 1005 in open position.

FIG. 22B. A close in top-down view of an embodiment of an oval dilation retractor 1005 in open position.

FIG. 22C. A perspective view of an embodiment of an oval dilation retractor 1005 in open position.

FIG. 23A. A top-down view of an embodiment of an oval dilation retractor 1005 in open position.

FIG. 23B. A close in top-down view of an embodiment of an oval dilation retractor 1005 in open position.

FIG. 23C. A perspective view of an embodiment of an oval dilation retractor 1005 in open position.

FIG. 24A. A top-down view of an embodiment of an oval dilation retractor 1005 in open position with the proximal arm 1020 and the distal arm 1030 spread apart.

FIG. 24B. A close in top-down view of an embodiment of an oval dilation retractor 1005 in open position with the proximal arm 1020 and the distal arm 1030 spread apart.

FIG. 24C. A perspective view of an embodiment of an oval dilation retractor 1005 in open position with the proximal arm 1020 and the distal arm 1030 spread apart.

FIG. 25A. A top-down view of an embodiment of an oval dilation retractor 1005 in open position with the proximal arm 1020 and the distal arm 1030 spread apart.

FIG. 25B. A close in top-down view of an embodiment of an oval dilation retractor 1005 in open position with the proximal arm 1020 and the distal arm 1030 spread apart.

FIG. 25C. A perspective view of an embodiment of an oval dilation retractor 1005 in open position with the proximal arm 1020 and the distal arm 1030 spread apart.

The figures may depict embodiments of the invention as utilized at a particular level of the spine, however it is intended that embodiments of the invention may be utilized at any level of the spine, and in particular at any level of the lumbar spine (including but not limited to L1-L2, L2-L3, L3-L4, L4-L5 and L5-S1).

### DETAILED DESCRIPTION

At the heart of the present invention is a system and method for performing surgical interventions related to the lumbar spine through an approach traversing either anterior to the psoas muscle or through the anterior portion of the psoas muscle. This novel approach avoids many of the drawbacks associated with targeting the posterior anatomy of the spine during the surgical approach, including excessive muscle trauma, nerve damage and the associated high rates of patient complications.

The present inventors have devised a variety of novel solutions to minimize the previously-unsolved disadvantages associated with targeting the anterior anatomy of the spine. The novel surgical trajectory associated with embodiments of the invention allows a surgeon to mitigate the substantial risk of injury to the musculature and nerve structures surrounding the spine caused by lateral approaches targeting the posterior anatomy of the spine. Moreover, the instrumentation associated with the preferred embodiment of the present invention includes a multi-bladed retractor assembly 1000 system that, in contrast to prior art retractors, incorporates specific features and configurations that allow blades to push soft tissue from a generally anterior location in a generally posterior trajectory while mitigating the risk of trauma to soft tissue. The differentiated method steps associated with embodiments of the invention, which facilitate approaching the anterior portion of the spine, also solve many previously-unsolved disadvantages associated with lateral interbody fusion.

The preferred embodiment of the present invention derives from the present inventors' realization of the flawed prevailing surgical philosophy that a surgical approach to the spine should avoid a trajectory near sensory nerves such as the genitofemoral nerve (“GFN”). This philosophy derives from the general inability for surgeons to detect the sensory nerves inter-operatively by utilizing standard neuro-monitoring techniques.

The present inventors have recognized that instead of simply and universally avoiding the sensory nerves, improved surgical approaches with trajectories near the sensory nerves may generate substantial advantages when utilized with a carefully prescribed set of method steps. Embodiments of the invention are associated with method steps of a surgical approach near the sensory nerves that mitigate the risks of injury to such sensory nerves.

By incorporating techniques to mitigate damage to the sensory nerves rather than avoid such sensory nerves completely, the present inventors have recognized alternatives to the prevailing surgical approach corridors to the spine. The techniques and instrumentation associated with embodiments of the invention allow for improved surgical interventions by utilizing a trajectory that traverses generally anterior to the psoas muscle or through the anterior portion of the psoas muscle, with a target point at the anterior third of the interbody disc space, while retaining the benefits of a substantially orthogonal, or lateral, approach to the spine.

Embodiments of the invention incorporate apparatuses to create and expand a working channel traversing through the skin to the spine on a trajectory anterior to the psoas muscle, or through the anterior portion of the psoas muscle. The present inventors recognize that the anterior portion of the psoas muscle is smaller than other portions of the psoas muscle, thus at or near its anterior portions there is less muscle tissue at risk of damage. Also, at or near the anterior portions of the psoas muscle, there generally are less nerves present than at or near other areas of the psoas muscle. Therefore, a trajectory through or near the anterior portion of the psoas muscle offers advantages over prior art trajectories through other areas of the psoas muscles by mitigating the risk of damage to muscle and nerve tissues.

The steps associated with creating a working channel through or near the anterior areas of the psoas muscle is accomplished in embodiments of the invention by placing a sequential series of dilators. In the preferred method of use, a first cannulated dilator is placed by a surgeon through an incision in the skin to the spine to the surface of the targeted disc space portion of the spine, on a path located anterior to the psoas muscle, or alternatively through the anterior portion of the psoas muscle.

In embodiments of the invention, the first cannulated dilator incorporates atraumatic features and specific dimensions. The atraumatic features are specifically designed to avoid damage to the soft tissues encountered during the approach trajectory anterior to the psoas muscle. In embodiments of the invention, these atraumatic features include rounded and chamfered edges that mitigate the risk of tissue or nerve damage during placement. In the preferred embodiment, the first cannulated dilator comprises high strength aluminum alloy. In embodiments of the invention, the diameter of the first dilator is 6-10 mm in diameter and 20-30 cm in length with a 3-4 mm cannulation.

After placement of the first cannulated dilator, a guide wire is placed through the cannula of the atraumatic cannulated dilator, the first dilator placed through the oblique and psoas tissues into the disc space. The preferred embodiment of the invention also incorporates a second dilator with an outer diameter of approximately 13-17 mm. In varying embodiments, the second dilator may take a substantially round or cylindrical profile (referred to herein as a “second round shaped dilator”) or may take an oval shaped profile (referred to herein as an “oval shaped dilator”). The present inventors have recognized separate and distinct advantages for optionally utilizing either a second round shaped dilator or second oval shaped dilator. Notably, the second round shaped dilator allows for further atraumatic expansion of the working channel by sequential placement of one or more additional dilators over the second round shaped dilator, as the round shape of the dilator lacks any narrow rounding points likely to damage the soft tissues. Separately, the oval shaped dilator allows for improved retraction of the soft tissues in combination with rotation of the dilator, leading to less trapping of soft tissues near the distal end of the dilator. In the preferred method of use, the second dilator, whether a second round shaped dilator or an oval shaped dilator, is slidably placed over the first cannulated dilator to push the surrounding soft tissues outward in a radial direction.

The preferred embodiment of the invention, with utilization of the second round shaped dilator, also incorporates a final dilator sequentially placed after placing the second round shaped dilator with an external diameter of 19-24 mm. In the preferred method of use, the final dilator is slidably placed over the second dilator to push the surrounding soft tissues outward in a radial direction. The retractor blades 1100 of the multi-bladed retractor assembly 1000, configured in substantially tubular form of a united blade construct having an internal diameter of approximately 20-26 mm in the preferred embodiment of the invention, may then be placed over the final dilator after placement of the final dilator over the other dilators.

In an alternative embodiment of the invention, incorporating utilization of an oval shaped dilator instead of the second round shaped dilator, no further dilators are intended to be sequentially placed following the placement of the oval shaped dilator. An embodiment of the oval shaped dilator associated with the invention comprises the following dimensions: 17-23 mm diameter in the major axis and 8-12 mm diameter in the minor axis with a rounded profile. The rounded, oval profile and external smooth surfaces of the oval shaped dilator in an embodiment of the invention allow the surrounding soft tissues to slidably move along the external surface of the oval shaped dilator minimizing the risk of trauma. The preferred embodiment of the oval shaped dilator consists of an overall length less than the length of the atraumatic cannulated dilator.

The preferred embodiment of the invention features a multi-bladed retractor assembly 1000. The preferred embodiment of the multi-bladed retractor assembly 1000 incorporates retractor blades 1100, which in their compressed, unified form, have a diameter slightly larger than the final dilator and a shape that contours to the external surface of the final dilator. In this way, the compressed form of the blades of the multi-bladed retractor assembly 1000 may slidably move exterior to and along the external surface of the final dilator with minimal impact to the surrounding soft tissues.

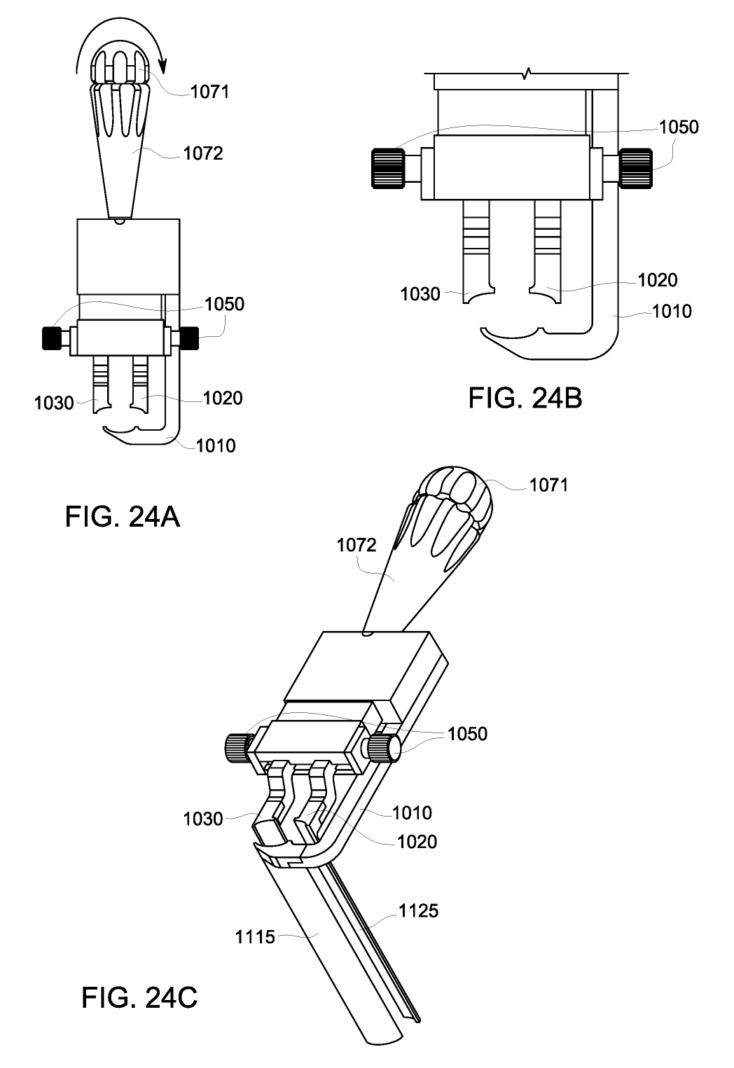

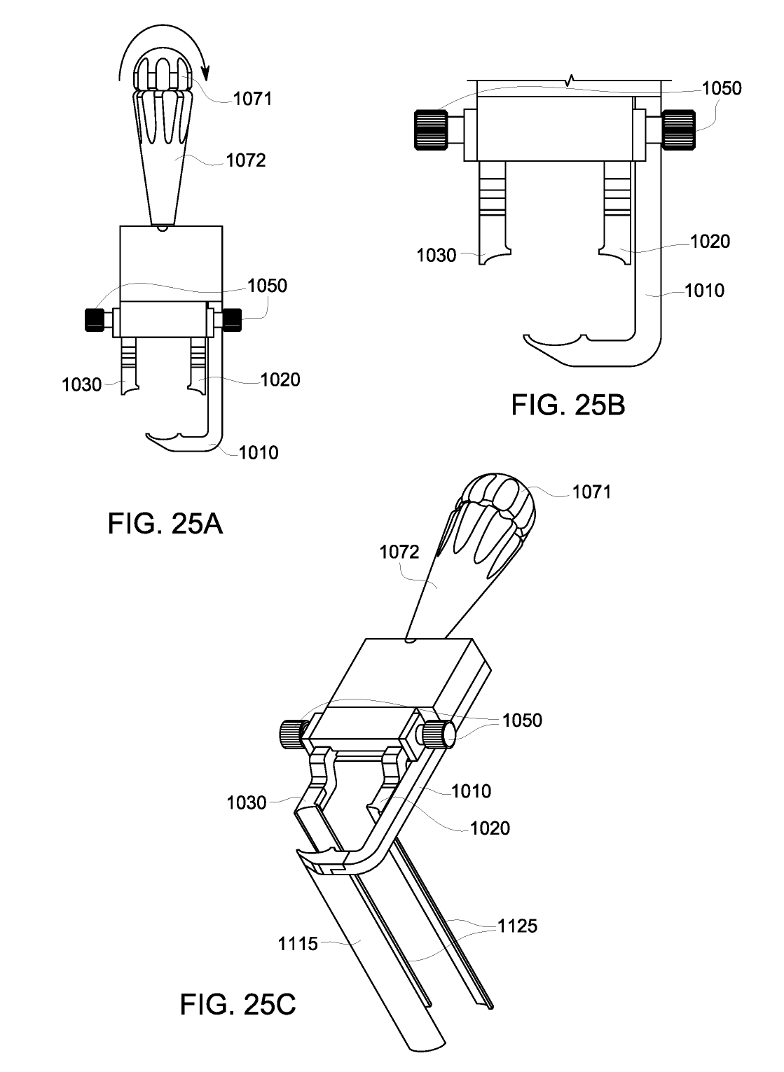

An embodiment of the invention incorporates an oval dilation retractor 1005. An oval dilation retractor 1005 in an embodiment of the invention is described as a multi-bladed retractor assembly able to hold a stationary retractor blade 1115 in position such that the distal end of the stationary retractor blade 1115 is docked within the anterior aspect of the disc space and allows for the movement of one or more movable retractor blades 1125 posteriorly in a direction opposite the stationary retractor blade 1115. In an embodiment, the oval dilation retractor 1005 houses a stationary retractor blade 1115 intended for docking in the anterior aspect of the disc space opposite two movable retractor blades 1125, wherein the stationary retractor blade 1115 is larger than each of the movable retractor blades 1125 as shown in FIG. 24C. An oval dilation retractor 1005 in an embodiment of the invention is also described as holding two or more one or more movable retractor blades 1125 with one or more mechanisms to independently move each of the movable retractor blades 1125 in a cephalad or caudal direction.

An embodiment of the invention incorporates one or more retractor arms 1003. In an embodiment, the retractor arms 1003 comprise a curved stationary arm 1010 and one or more movable arms. In an embodiment, the movable arms consist of a proximal arm 1020 and a distal arm 1030. As used herein, the term “proximal arm” refers to arm located nearest to the curved stationary arm 1010. As used herein, the term “distal arm” refers to the arm farthest from the curved stationary arm 1010. In an embodiment, each of the retractor arms is configured to hold a retractor blade 1100.

In an embodiment, the multi-bladed retractor assembly 1000, optionally consisting of an oval dilation retractor 1005, is capable of transitioning from an anterior to posterior primary retraction facilitator to a traditional posterior to anterior retraction facilitator by changing the orientation of the multi-bladed retractor assembly 1000. In such an embodiment, the retractor blades 1100 are removable and placed orthogonally into the retractor arms on the opposite side. In such configuration, the multi-bladed retractor assembly 1000 is flipped in orientation allowing for the user to dock the stationary retractor blade 1110 in a posterior position and then retract the one or more movable blades 1125 in an anterior direction to open a working channel. In such embodiment, the retractor arms are configured with apertures opening in opposing directions such as to hold a retractor blade oriented in either direction orthogonal to the trajectory of the arm itself.

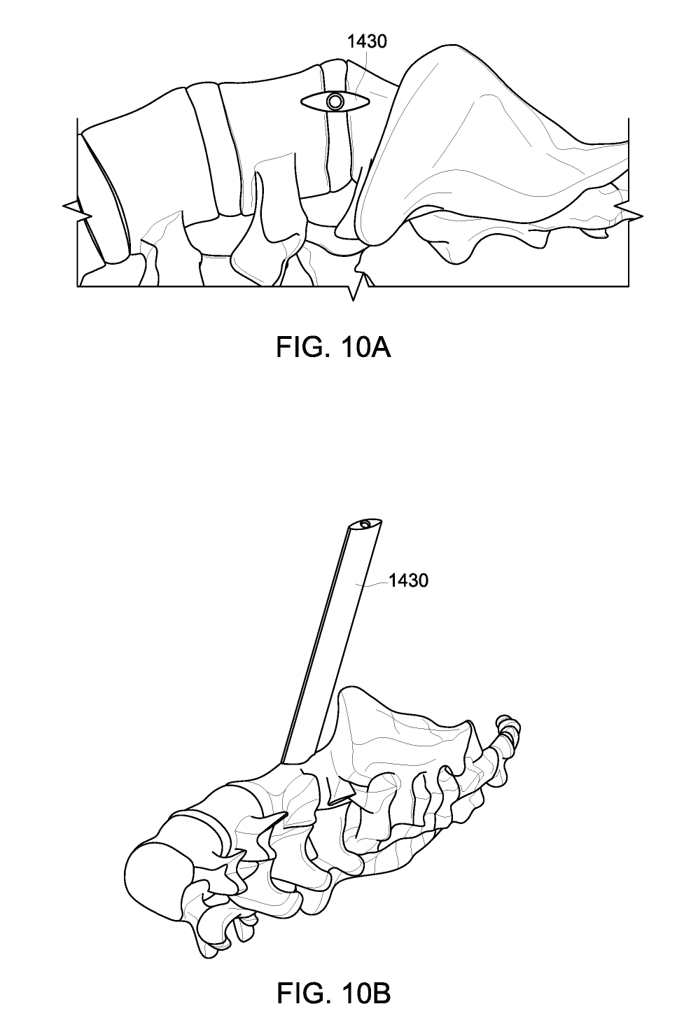

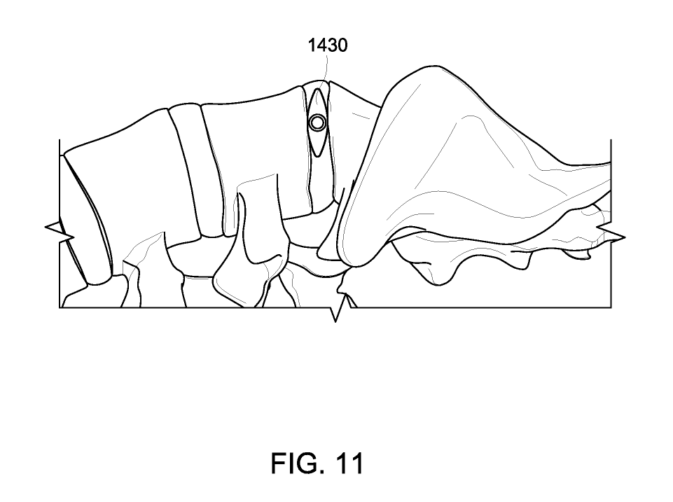

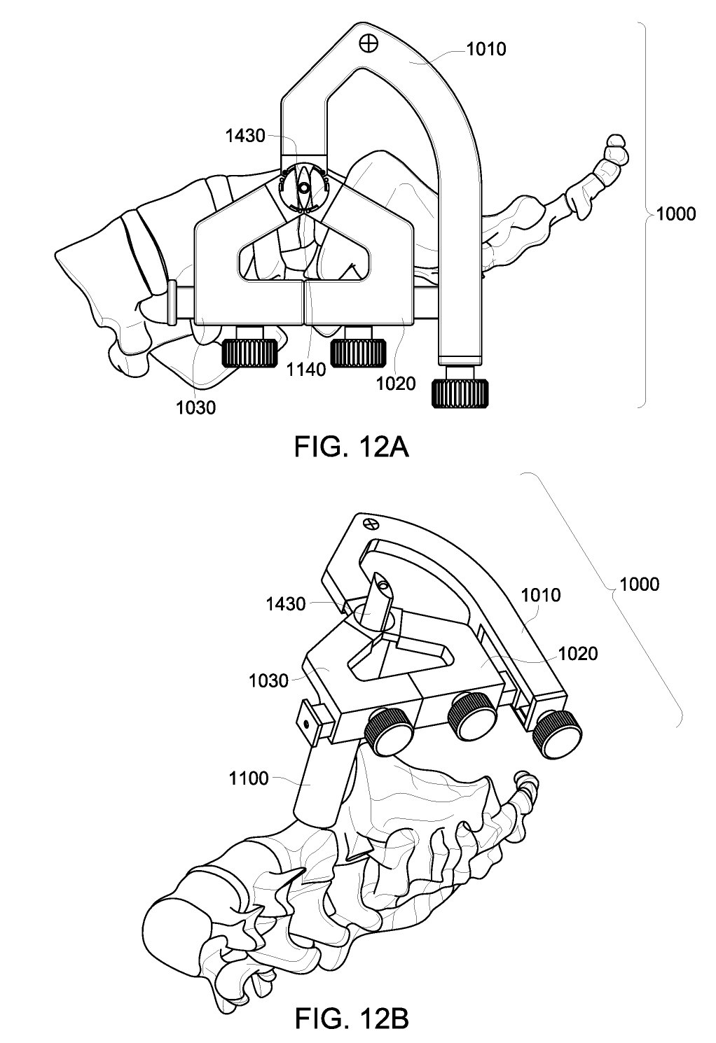

In an embodiment, the curved stationary arm 1010 is intended to remain in a fixed position relative to the spine following placement. In an embodiment, the curved stationary arm 1010 is configured to allow for the positioning of a portion of the multi-bladed retractor assembly 1000 away from the user's field of view during use. In an embodiment, the curved stationary arm 1010 serves as the connection point for the stationary retractor blade 1110 to the multi-bladed retractor assembly 1000. In an embodiment, the curved stationary arm 1010 resembles an “L” or a “J” shape to move a significant portion of the mass of the multi-bladed retractor assembly 1000 away from the user's field of view during surgery. FIG. 10A depicts the curved stationary arm 1010 in a modified “J”-shape configuration.

An embodiment of the invention incorporates a movement actuator 1050. The movement actuator 1050 in an embodiment incorporates a knob intended to be turned by a user. The movement actuator 1050 functions to move either the proximal arm 1020 or the distal arm 1030 independently from the other. In an embodiment, assuming placement of the retractor such that the stationary retractor blade 1110 is located in an anterior position relative to the spine as shown in Fig. X, the movement actuator 1020 functions to move the blades affixed to a movable arm in either a caudal or cranial direction. The movement actuator 1020 functions via a worm gear, a rack and pinion or a threaded drive shaft in varying embodiments of the invention.

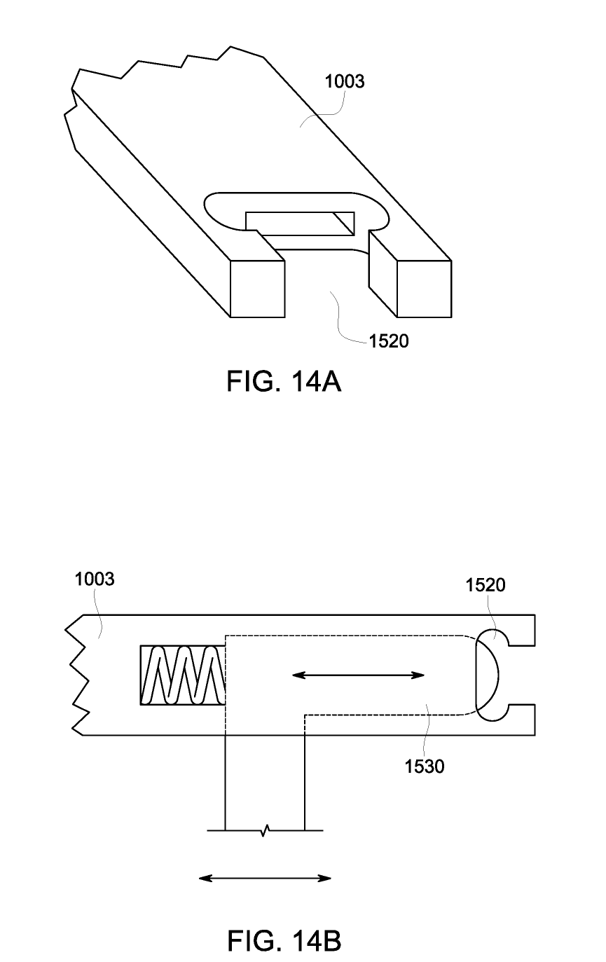

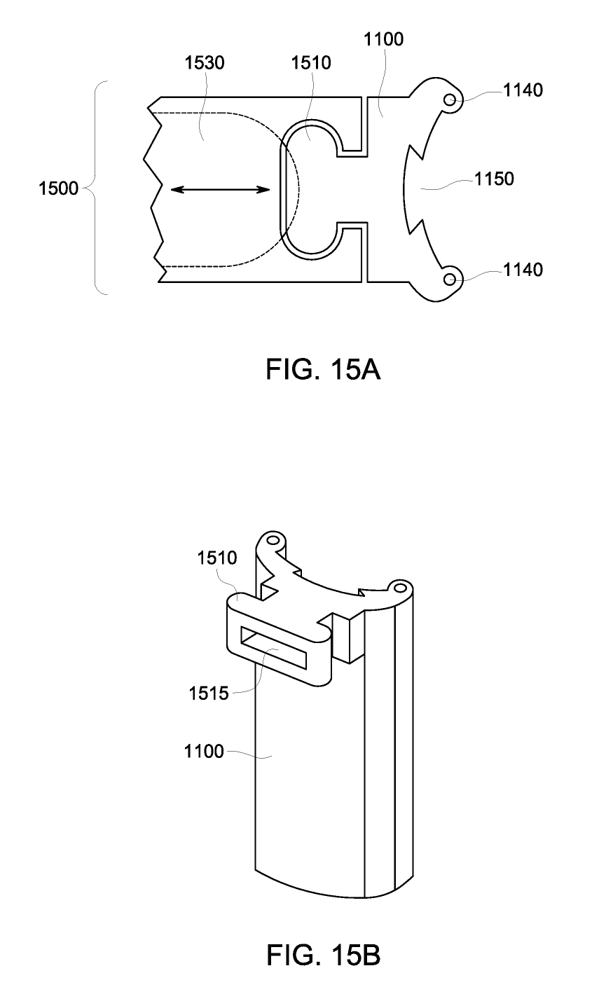

An embodiment of the invention incorporates one or more retractor blades 1100. In an embodiment of the invention, the retractor blades 1100 are affixed to the retractor 1000 via a set screw. Although described herein as using a set screw to secure the retractor blades 1100 to any or all of the retractor arms, which may include one or more of the stationary arm 1010, the proximal arm 1020 or the distal arm 1030, any suitable attachment mechanism can be used without departing from the scope of the present invention. For example, a quick connection mechanism such as a snap fit engagement is possible, as is a friction engagement or an integral blade. In an embodiment, a quick connect 1500 is incorporated as depicted in FIG. 14 and FIG. 15. The one or more retractor blades 1100, which optionally may include a stationary retractor blade 1110, and one or more movable retractor blades 1125, may be provided in any size or shape suitable to establish and maintain an operative corridor to the surgical target site. By way of example only, in an embodiment, a retractor blade 1100 includes an attachment portion and a blade portion. In an embodiment of the invention, the attachment portion comprises a male retractor blade connect protrusion 1510 as depicted in FIG. 15A and FIG. 15B. In an embodiment, the male retractor blade connect protrusion 1510 incorporates a protrusion engagement slot 1515. In an embodiment, the retractor blade 1100 incorporates a locking mechanism. In an embodiment, the locking mechanism comprises a protrusion engagement slot 1515 allows for the receipt of a bolt-like mechanism pressed by a quick connect compressive mechanism 1530 at the opposite end. The attachment portion is configured to fit within an aperture of either the stationary arm 1010, the proximal arm 1020 or the distal arm 1030. In an embodiment, the aperture is configured as a female quick connect receptacle 1520 as depicted in FIG. 14A and FIG. 14B. In an embodiment, the aperture is configured to receive the attachment portion of a retractor blade 1100. In varying embodiments, each aperture is integrated into either the stationary arm 1010, the proximal arm 1020 or the distal arm 1030. The engagement between the retractor blade 1100 and either the stationary arm 1010, the proximal arm 1020 or the distal arm 1030 is provided by way of example as a snap-fit engagement allowing for relative easy insertion and/or removal of the retractor blade 1100. However, other engagements are possible without departing from the scope of the present invention, including but not limited to using a set screw (e.g. through either the stationary arm 1010, the proximal arm 1020 or the distal arm 1030 into the aperture), friction engagement, or providing a medial retraction member with integral blade. This engagement allows a user to intraoperatively change the retractor blade 1100, for example to swap out a shorter blade for a longer blade, and vice versa. In alternative embodiments, the aperture of a retractor arm is bi-directional, enabling a user to place a retractor blade 1100 in either side of the aperture, which allows the user to flip the multi-bladed retractor assembly 1000 in a manner such that the user can retract tissue in either a posterior to anterior trajectory or in an anterior to posterior trajectory.

An embodiment of the invention incorporates one or more retractor blades 1100. In an embodiment of the invention, one or more of the retractor blades 1100 incorporates a blade slot 1150. The blade slot 1150 is configured to slideably receive a shim attachment, for example the safety barrier shim 1160 shown and described herein. It should be understood, however, that any suitable shim attachment may be used without departing from the scope of the present invention. In an embodiment of the invention, at the distal end of each of the blade slots 1150 there is a stop, which interacts with the shim to prevent the shim from passing the stop once it has been fully engaged to the retractor blade. In an embodiment, the blade slot 1150 incorporates a first lip and second lip. The first lip and second lip comprise edges of the retractor blade adjacent to the blade slot and each extend along the length of the retractor blade. The first lip and second lip interact with the geometry of the shim in an embodiment of the invention to contain the shim while enabling the shim to slidably move along the length of the retractor blade.

An embodiment of the invention incorporates a stationary retractor blade 1110. In an embodiment, the stationary retractor blade 1110 is configured for attachment to stationary arm 1010.

An embodiment of the invention incorporates a toeing actuator 1060. In an embodiment, the toeing actuator 1060 functions to allow a user to toe a retractor blade 1100. In an embodiment, the toeing actuator 1060 incorporates a knob to receive rotational force from a user. In an embodiment the toeing actuator 1060 incorporates a threaded drive shaft to move a retractor blade 1100. In an alternative embodiment, the toeing actuator 1060 incorporates a rack and pinion to move a retractor blade 1100. In an embodiment, the toeing actuator 1060 rotates a retractor blade around a toeing hinge 1040 as the pivot point. In an embodiment of the invention, the toeing hinge 1040 is located at point where the proximal end of the retractor blade meets the retractor arm.

An embodiment of the invention incorporates an actuator handle 1070. The actuator handle 1070 protrudes from the main body of the retractor 1000 providing a grip for the user. In varying embodiments, the actuator handle 1070 incorporates an end knob 107, a rotating arm actuator 1072, or both.

An embodiment of the invention incorporates an end knob 1071. In an embodiment, user interaction with the end knob 1071 controls the movement of the blades affixed to the proximal arm 1020 and distal arm 1030 simultaneously in either an anterior or posterior direction (assuming placement of the retractor such that the stationary retractor blade 1110 is located in an anterior position or posterior position relative to the spine). In an embodiment, the end knob 1071 actuates the movement of the movable blades together in a direction orthogonal to the stationary blade 1110. In an embodiment, turning of the end knob 1071 facilitates movement of the blades affixed to the proximal arm 1020 and distal arm 1030 via a worm gear mechanism. In alternative embodiments, the end knob 1071 facilitates movement of the blades affixed to the proximal arm 1020 and distal arm 1030 via a threaded drive shaft or rack and pinion mechanism.

In an embodiment, the rotating arm actuator 1072 functions to move the proximal arm 1020 and the distal arm 1030 symmetrically. In an embodiment, the symmetrical movement of the proximal arm 1020 and the distal arm 1030 is accomplished via a rack and pinion mechanism. Thus, as the rotating arm actuator 1072 is turned by a user, a gear causes a first and second racks correspondingly associated with the proximal arm and the distal arm 1030 to simultaneously move in opposite directions. For example, when the rotating arm actuator 1072 is rotated in a clockwise direction, the first rack will move the proximal arm 1020 in a caudal direction (assuming placement of the retractor such that the stationary retractor blade 1110 is located in an anterior position relative to the spine) and the second rack will move the distal arm 1030 in a cranial direction. The effect of this movement is that a first movable retractor blade 1125, through its connection to the proximal arm 1020 (which is connected to the first rack member) will move in a caudal direction and a second movable retractor blade 1125, through its connection to the distal arm 1030 (which is connected to the second rack member) will move simultaneously in a cranial direction. Alternatively, the user can configure the invention such that the stationary retractor blade 1110 is located in a posterior position relative to the spine, whereby the directional movements actuated by turning the rotating arm actuator 1072 are reversed. In an embodiment, the rotating arm actuator 1072 incorporates a worm gear mechanism to facilitate the movement of either or both the proximal arm 1020 and the distal arm 1030. In an embodiment, the mechanisms actuated via turning the rotating arm actuator 1072 operates in conjunction with independent worm gears associated with either the proximal arm 1020 or the distal arm 1030, thereby allowing the user to independently move the one or more retractor blades 1100 attached to the proximal arm 1020 or distal arm 1030, either independently via the movement actuator 1050 controlling the independent worm gears associated with either the proximal arm 1020 or the distal arm 1030, or together symmetrically via the rotating arm actuator 1072.

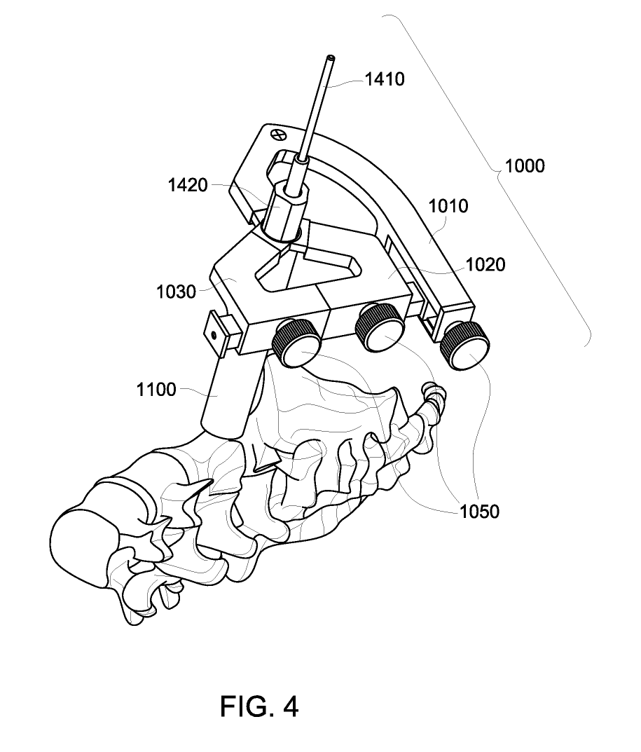

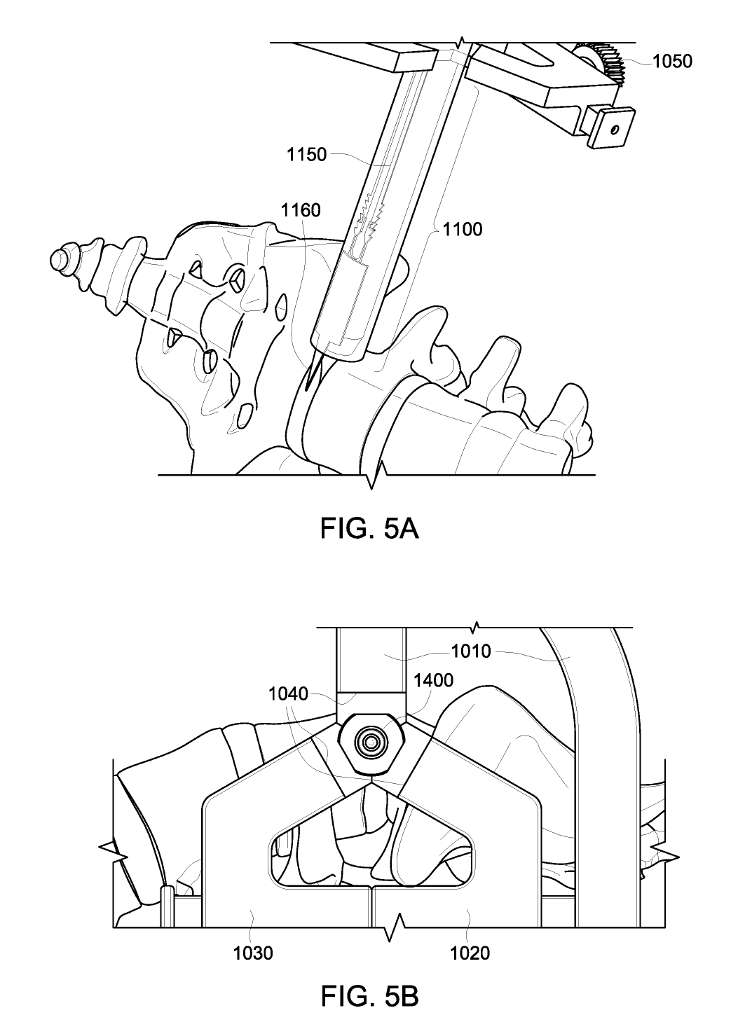

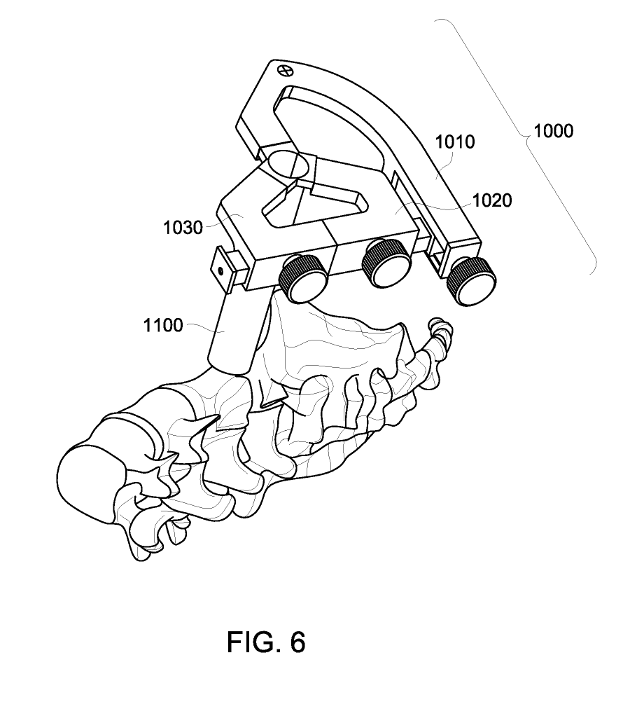

An embodiment of the invention incorporates a safety barrier shim 1160. In an embodiment, the safety barrier shim 1160 is a generally rectangular elongated member. The safety barrier shim 1160 in an embodiment includes a flat extension extending substantially the length of one side. In an embodiment, the flat extension is dimensioned to engage one of the blade slot 1150 of a retractor blade 1100) to enable slidable engagement of the safety barrier shim 1160 with a retractor blade 1100, as shown in FIG. 7. In an embodiment, the safety barrier shim 1160 further includes a recess formed within its back side. The recess is dimensioned to receive at least a portion of a flange incorporated into the retractor blade 1100 to transition to a locked position. In an embodiment of the invention, the present inventors have recognized an advantage that the safety barrier shim 1160 may be positioned into place in the body while the one or more dilators are enclosed by the stationary retractor blade and the at least one movable retractor blade in the closed position. In an embodiment, this is due to the hexagonal shape of the final dilator 1420, as depicted in FIG. 4, which provides space for the safety barrier shim 1160 to be slid over the final dilator 1420 while the final dilator 1420 is in place.

In an embodiment of the invention, the multi-bladed retractor assembly 1000 incorporates a main retractor body. In an embodiment of the invention, the main retractor body comprises the general framework and mechanical structure of the retractor. One skilled in the art recognizes that all mechanical motion and advantage results from this structure. In an embodiment, the main retractor body comprises the curved stationary arm 1010. The table mounted retractor arm (TMRA) in embodiments of the invention is an articulating device that provides a mechanism of attaching and fixating the retractor body to the operating table. Embodiments of the TMRA attach to the retractor with various mechanisms, but in the preferred embodiment of the invention the mechanism of attachment to the retractor optionally takes place with either a ball detent quick connect or a wing nut and screw connection. In the preferred embodiment of the invention, the attachment point of the TMRA to the retractor is on the side opposite the surgeon, near the anterior of the patient. In the preferred embodiment, the TMRA connects to standard rails on the table with a vise connection over the rail.

The preferred embodiment of the multi-bladed retractor assembly 1000 is configured to optionally allow for the retraction of soft tissues by pushing such soft tissues from a substantially anterior position in a posterior direction to a substantially posterior position relative to their natural position. The multi-bladed retractor assembly 1000 posterior-directional retraction mechanism is enabled by portions of the multi-bladed retractor assembly 1000 docking with the anatomy of the spine in an anterior position. In the preferred embodiment of the invention, a safety barrier shim 1160, affixed to the stationary retractor blade 1110, forms the distal-most protrusion that docks with the anterior aspect of the disc space. In alternative embodiments of the invention, stationary retractor blade 1110 itself without the safety barrier shim 1160 forms the protrusion that docks with the anterior aspect of the disc space.

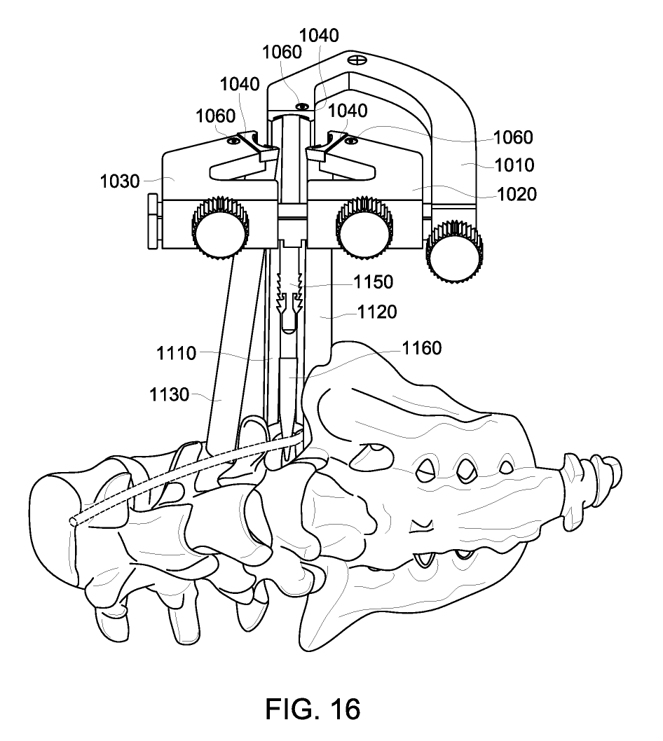

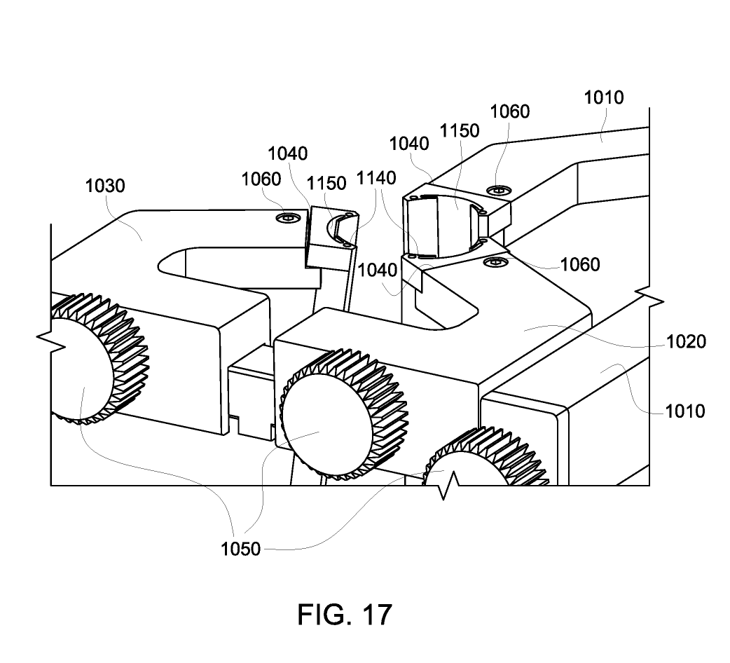

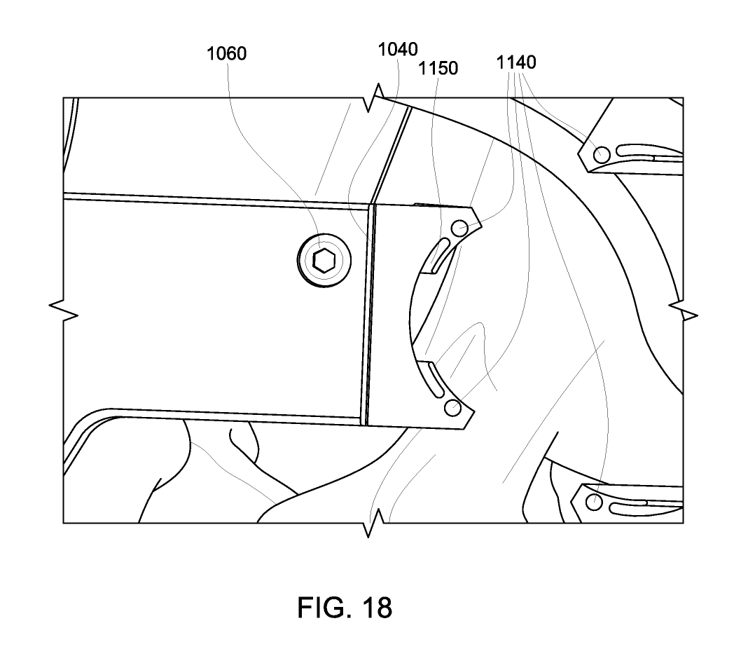

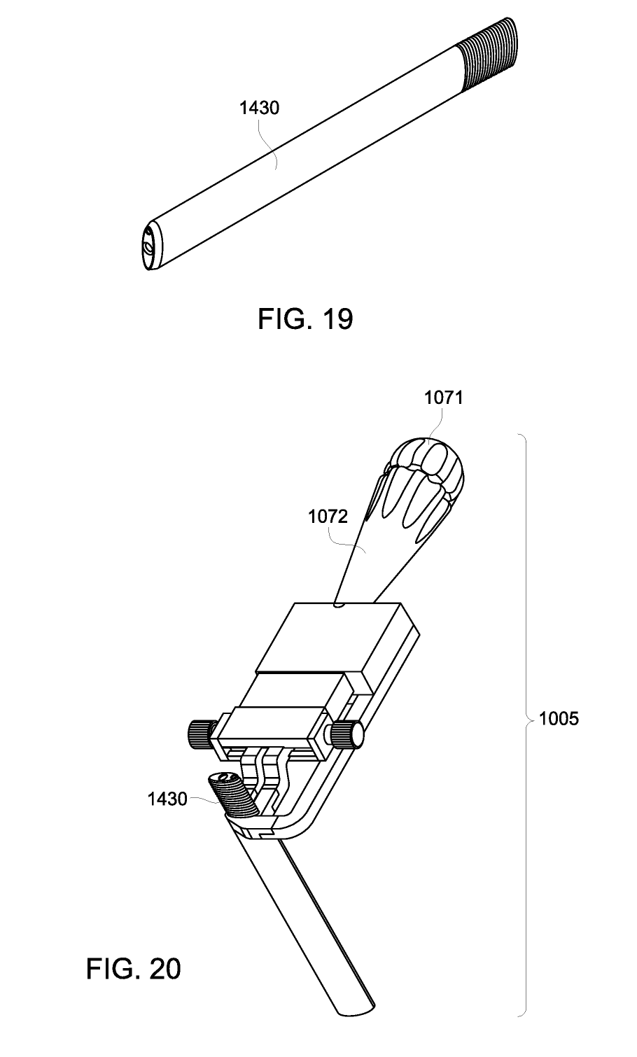

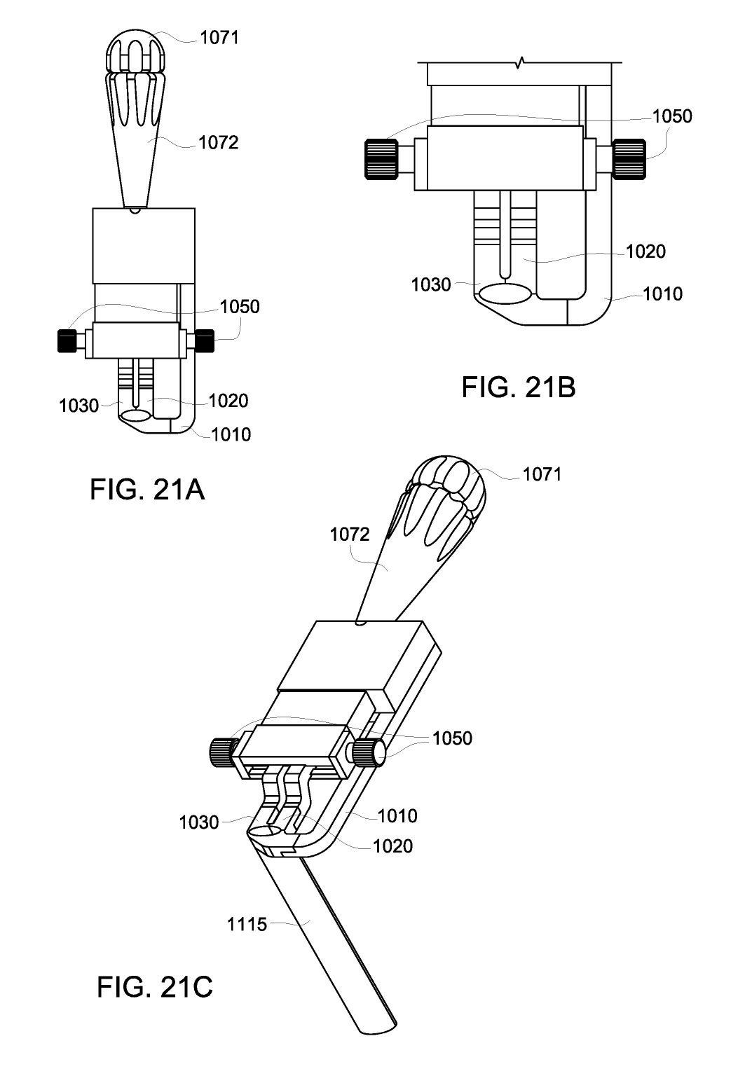

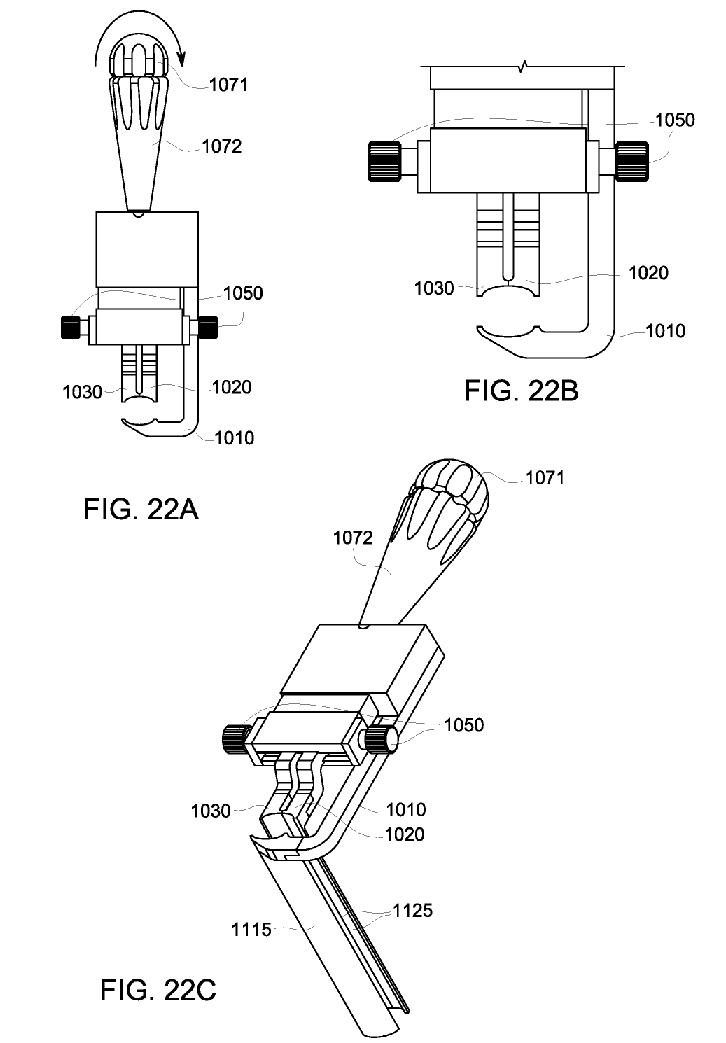

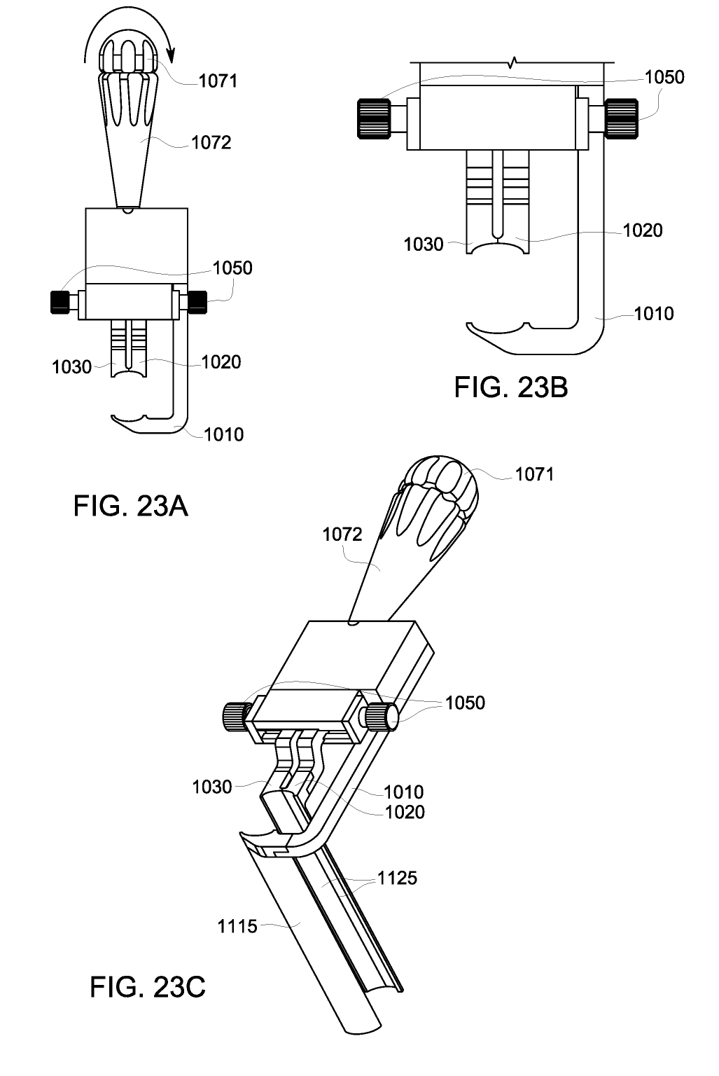

Varying embodiments of the retractor incorporate two or more retractor blades 1100. In the preferred embodiment of the invention, the retractor incorporates three retractor blades 1100, consisting of a proximal blade 1120, a distal blade 1130 and a stationary retractor blade 1110, as depicted in FIG. 16. The retractor blades 1100 extend from orthogonally from the retractor arms to enable enclosure of a dilator. In a two-bladed embodiment of the multi-bladed retractor assembly 1000, the proximal blade 1120 and the distal blade 1130 are replaced by a single blade, opposing the stationary blade 1110. In such embodiment, the single movable retractor blade travels only in either an anterior or posterior trajectory, with no independent movement in either a caudal or cephalad trajectory. The retractor blades 1100, once expanded, create a portion of the boundary of a surgeon's working channel. In an embodiment, the dimensions of a working channel, also variously referred to as an “operating corridor” or “distraction corridor along the lateral path to the lumbar spine” are described in United States Patent Application having Publication Number US 2010/0069783 A1. U.S. patent application 12/623,016 with Publication Number US 2010/0069783 A1 filed in the United States Patent and Trademark Office with a filing date of Nov. 20, 2009 is hereby incorporated by reference.

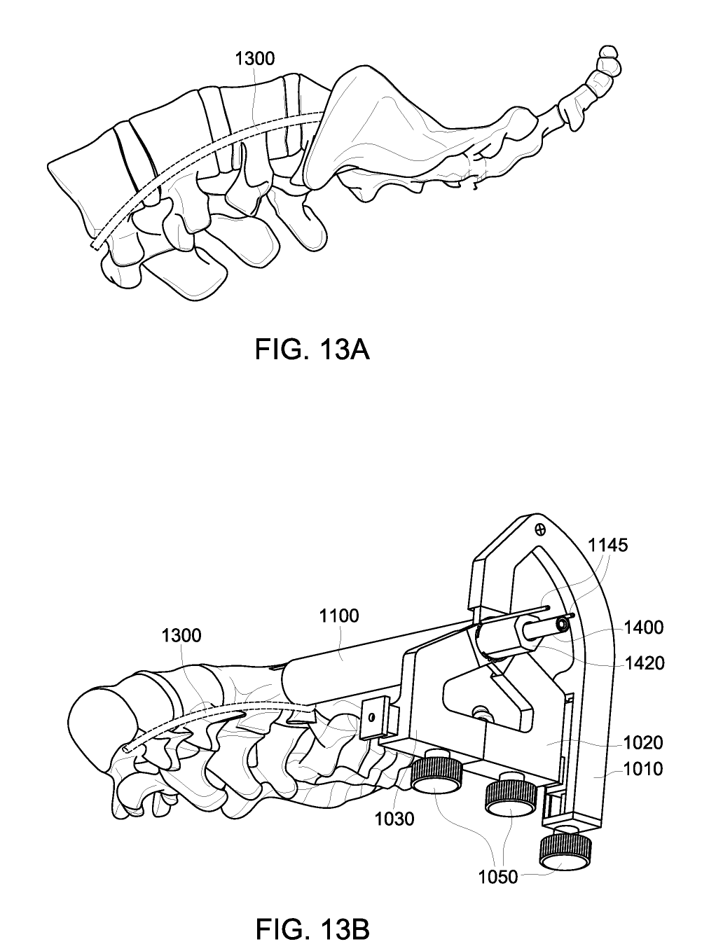

In varying embodiments of the invention, of the retractor blades 1100, one retractor blade is a stationary retractor blade 1110. The remaining retractor blades consist of movable retractor blades 1125. In an embodiment, at least one of the movable retractor blades 1125 is configured to have the ability to move in a generally posterior direction relative to the anteriorly placed stationary retractor blade 1110. In an embodiment, the movable retractor blade's movement, either independently from or in concert with the other movable retractor blades, in a generally posterior direction thereby pushes the adjacent soft tissues in a generally posterior direction. The present inventor has recognized that the adjacent soft tissues include a sensory nerve 1300, as depicted in FIG. 13A. The present inventor also recognizes that the sensory nerve 1300, though unlike the motor nerve is difficult to detect with conventionally known neuromonitoring techniques utilized in association with other systems for approaching the spine laterally, is generally more resilient and less susceptible to damage than the more fragile and rigid motor nerve. Moreover, the present inventor has recognized that the orientation of the sensory nerve along the spine is such that the anterior to posterior retraction such as that accomplished by the preferred embodiment of the multi-bladed retractor assembly 1000 disclosed herein moves the sensory nerve 1300 in such a way that it does not stretch or elongate the sensory nerve 1300. Thus, the present inventors have devised an apparatus that when used as intended in its preferred embodiment enables the avoidance of damage to the sensory nerve 1300.

In embodiments of the invention, once the stationary retractor blade 1110 is in place and fixated, the surgeon may then advance the movable retractor blades 1125 generally in the posterior direction. Such movement may be performed optionally by using either mechanically guided motion from the surgeon, a ratchet and pawl mechanism, or a drive screw mechanism. In embodiments of the invention in which the multi-bladed retractor assembly 1000 comprises at least three retractor blades, the at least two movable retractor blades comprise at least one movable retractor blade in a generally cephalad orientation relative to the other movable blade(s) and at least one movable retractor blade in a generally caudal orientation relative to the other movable blade(s). In embodiments of the invention, the multi-bladed retractor assembly incorporates a ratchet and pawl mechanism or drive screw mechanism to facilitate movement of the retractor arms and attached retractor blades 1100. In such embodiments, the ratchet and pawl mechanism or drive screw mechanism of the multi-bladed retractor assembly receive rotary power input from the surgeon and output linear motion with a mechanical advantage. The preferred embodiment of the invention incorporates a locking mechanism to allow the surgeon to lock the retractor blades 1100 into position once the desired level of retraction is achieved. In varying embodiments, the locking mechanism comprises a spring lock as a part of the drive mechanism incorporated into the multi-bladed retractor assembly 1000. In an embodiment, the locking mechanism comprises a lock nut as part of the drive mechanism.

In the preferred embodiment of the invention, the retractor blades 1100 are produced to a configuration such that when the retractor blades 1100 come together into a united configuration, when viewed as a whole, they resemble a substantially tubular form with a circular cross-sectional shape. In an alternative embodiment of the invention, the retractor blades 1100 are produced to a configuration such that when the retractor blades 1100 come together into a united configuration, when viewed as a whole, they resemble a substantially tubular form with an oval cross-sectional shape. In embodiments of the invention, each of the retractor blades 1100 are curved with a radius of 10-12 mm. In embodiments of the invention, the retractor incorporates retractor blades 1100 with lengths in the range of 120 mm-200 mm. The present inventors specifically recognize the needs posed by a variety of anatomical variations, therefore embodiments of the invention are configurably accommodative of retractor blades 1100 with varying blade lengths to address differing anatomical variations. In embodiments of the invention, the diameter of the substantially tubular form of the united blade construct having a circular cross-sectional shape in the compressed configuration is 20-25 mm.

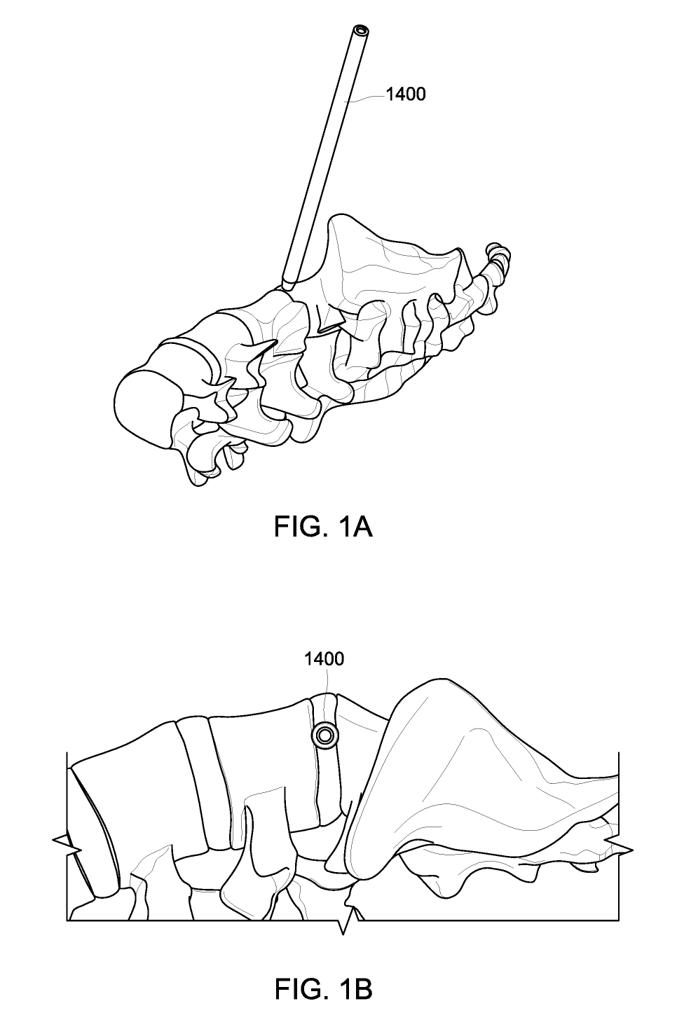

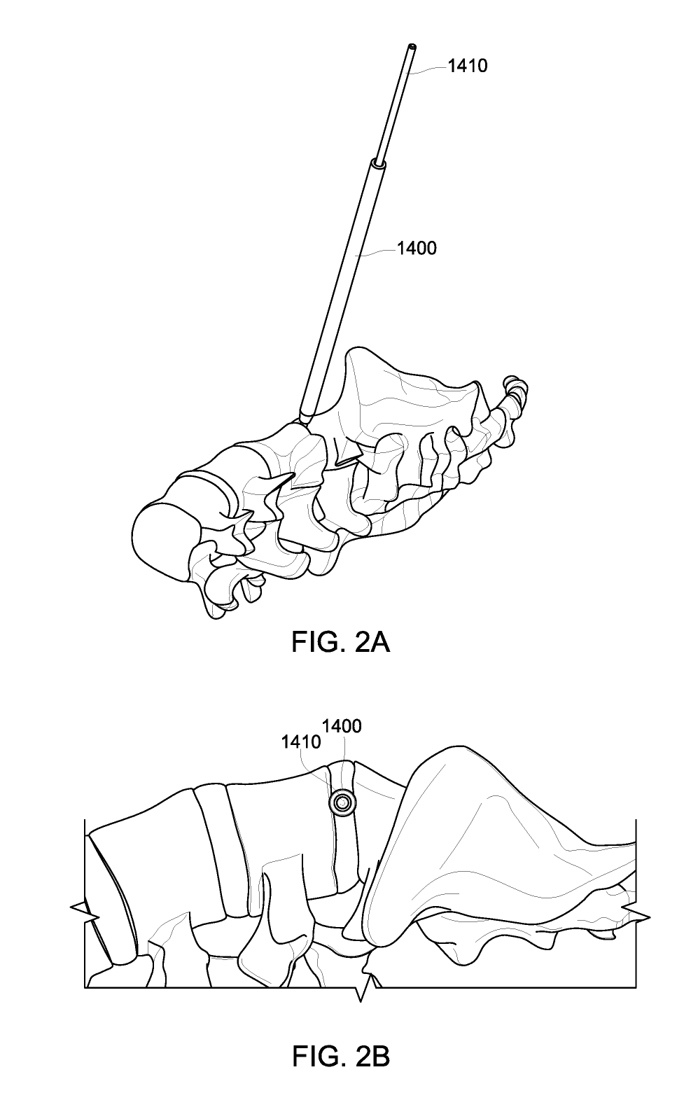

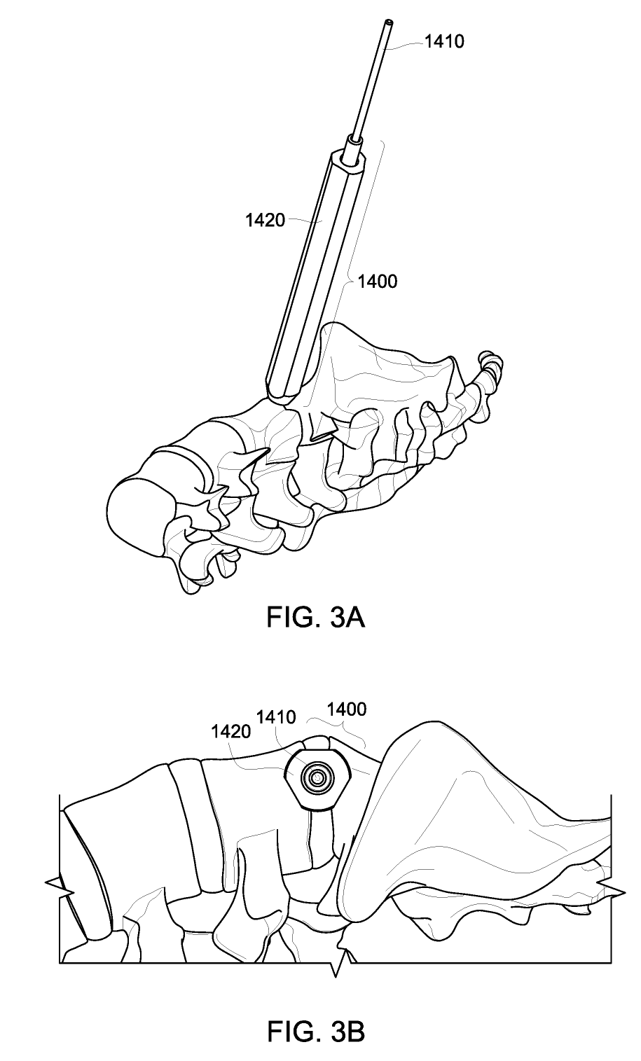

Embodiments of the invention incorporate a dilator system. In certain embodiments, the dilator system is configured to create a distraction corridor along the lateral path to the lumbar spine. In varying embodiments, the dilator system incorporates an elongate member 1410. In an embodiment of the invention, the one or more dilators associated with the dilator system incorporates a dilator cannula intended to slidably engage an elongate member 1410, as depicted in FIG. 3A. In an embodiment, the elongate member 1410 consists of a Kirschner Wire. In the preferred method of use, the user places the elongate member 1410 through the skin on a trajectory into the disc space to define the approach pathway. In such method, one or more subsequent dilators, including a final dilator 1420, are slid over the elongate member 1410. In an embodiment, the final dilator 1420 incorporates a cross-sectional shape to accommodate for the passage of a blade slot 1150 incorporated into one or more retractor blades 1100 and/or the safety barrier shim 1160. In an embodiment, the cross section of the final dilator 1420 has dimensions approximating a hexagonal shape, as depicted in FIG. 3, to accommodate the passage of one or more retractor blades 1100 incorporating a blade slot 1150. In an embodiment, the dilators are configured as depicted in FIG. 1 A-B and FIG. 3 A-B. In an embodiment, the dilator system comprises the dilation mechanisms as described in U.S. patent application 12/623,016 with Publication Number US 2010/0069783 A1 filed in the United States Patent and Trademark Office with a filing date of Nov. 20, 2009, which is hereby incorporated by reference. In varying embodiments, the diameter of the substantially tubular form of the united blade construct is only slightly larger than the diameter of the final dilator 1420, such that the united blade construct is slidably movable over the final dilator to result in minimal disruption to the soft tissues external to and immediately surrounding the final dilator 1420 during and after placement.

In varying embodiments of the invention, the cross-sectional form of the united blade construct may take a substantially oval shape. In this configuration, the retractor blades may move slidably along an element of the dilator system comprising an oval shaped dilator 1430 as depicted in FIG. 10B. In an embodiment of the invention, the oval shaped dilator 1430 incorporates a dilator cannula intended to slidably engage an elongate member 1410. The present inventor has recognized that an oval shaped dilator 1430 allows a surgeon to place the elongated aspect of the oval shaped dilator 1430 parallel to muscle fibers in a manner that minimizes disruption to the psoas muscle and surrounding tissues. The present inventors have recognized that another advantage of the oval shape profile in both the retractor blades in the form of united blade construct and in the oval shaped dilator 1430 associated with varying embodiments of the invention, generally, is that the oval profile along with the rotation of the dilator step in association with embodiments of the invention operates to minimize the trappage or pinching of soft tissues at points where such soft tissues are in contact with the oval shaped 1430 dilator and retractor blades 1100. The present inventors have recognized that minimization of the trappage or pinching of soft tissue resulting from the oval shape profile applies especially with regard to dilation through the psoas muscle.

In embodiments of the invention, the disclosed apparatuses and techniques associated with docking the multi-bladed retractor assembly 1000 with the anatomy of the spine in an anterior position allow for the establishment of a protective boundary. In the preferred embodiment of the invention, the safety barrier shim 1160 protective boundary shields the tissues generally to the anterior of the protective boundary from the implants and instrumentation passed by the surgeon generally to the posterior of the protective boundary. The present inventors have recognized that a surgeon may create such protective boundary by advancing the safety barrier shim 1160 along the stationary retractor blade 1110 and inserting it into or proximal to the anterior aspect of the disc space, generally posterior to the anterior longitudinal ligament, as depicted by FIG. 7. The present inventors have recognized that such protective boundary forms a barrier between the great vessels and other bodies anterior to the approach trajectory. Thus, the working channel along the approach trajectory, which proceeds into and including what one skilled in the art would recognize as zones 1 and 2 of the disc space, is shielded anteriorly from structures with which inadvertent contact could cause great harm. The protective boundary associated with the embodiments of the apparatuses and techniques disclosed herein is established by a surgeon placing a portion of the retractor or attachment to the retractor, such as the safety barrier shim 1160, either into or immediately posterior to the anterior longitudinal ligament (ALL), which traverses the spinal column in a cephalad-caudal direction, to dock the retractor and create a protective boundary between the working channel and bodies anterior. In certain embodiments, the surgeon may choose to place a portion of the retractor or attachment to the retractor such as the safety barrier shim 1160 between the ALL and the anterior face of a vertebral body to dock the retractor and create a protective boundary between the working channel and bodies anterior. When properly docked, the preferred embodiment of the multi-bladed retractor assembly 1000, when utilized in association with the method steps contemplated by the inventors to retract in a generally posterior direction, creates an aperture forming a working channel along a substantially lateral surgical approach generally targeting the anterior third of the disc space.

The stationary retractor blade 1110 in embodiments of the invention incorporates a retractor blade slot. The retractor blade slot exists on the interior face of the stationary retractor blade. In the preferred embodiment, the retractor blade slot has a width of 15 mm and runs substantially the entire length of the stationary retractor blade. The blade slot 1150 is designed to attachably accommodate a safety barrier shim 1160 within the stationary blade 1110. The blade slot 1150 interacts with a protrusion of the safety barrier shim 1160 to enable the safety barrier shim 1160 to movably slide along the length of the stationary blade 1110 in a controlled and constrained manner. A novel aspect of the stationary blade 1110 associated with the preferred embodiment of the invention is that the exterior face of the stationary blade 1110 is orientable away from the surgeon without other portions of the retractor assembly interfering with the user's (typically a surgeon's) field of view.

The preferred embodiment of the invention features a safety barrier shim 1160 contoured to enable it to immovably dock near the anterior third of the interbody space, either within or immediately posterior to the anterior longitudinal ligament (ALL). In embodiments of the invention, the cross-section of the safety barrier shim 1160 comprises a substantially wedge-like profile. In the preferred method of use, the safety barrier shim 1160 is tapped into the soft tissue of the disc space as far as possible at a depth of not more than 25 mm. In varying embodiments of the invention, the surgeon may controllably deploy the safety barrier shim 1160 at any depth up to 25 mm. In varying embodiments, the surgeon may retract deployment of the safety barrier shim 1160 at any point during the procedure. Varying embodiments of the invention incorporate a ratchet feature on the blade and a pawl feature on the safety barrier shim 1160 to prevent it from backing out until desired.

In the preferred method of use, the placement of the safety barrier shim 1160 creates a barrier between the working channel and the anterior structures. For purposes herein, the term “working channel” refers to the area generally between the retractor blades in their opened state. In the preferred method of use, such the safety barrier shim 1160 is intended create a barrier between the working channel and the structures to the anterior to the safety barrier shim 1160, which include all or part of the anterior longitudinal ligament, the vena cava and the aorta. The present inventor has recognized a particular benefit of the safety barrier shim 1160, namely the mitigation of risk of harm to the great vessels anterior to the safety barrier shim 1160. Thus, by placement of the safety barrier shim 1160, a safety zone is created posterior to the safety barrier shim 1160 within which a surgeon may pass instrumentation and/or implants while minimizing risk of inadvertent contact with structures anterior to the working channel. The present inventor has recognized that instrumentation and/or implants placed through and within the safety zone created posterior to the safety barrier shim 1160 will be shielded by the safety barrier shim 1160 from the great vessels.

In the preferred method of use, tapping the safety barrier shim 1160 into the disc space provides an anchoring point to the body for the multi-bladed retractor assembly 1000. In embodiments of the invention, the safety barrier shim is approximately 50-75 mm in length. In the preferred embodiment of the invention, the safety barrier shim comprises stainless steel. In the preferred embodiment of the invention, the safety barrier shim has a profile that matches the profile of the stationary retractor blade.

In the preferred method of use, the safety barrier shim 1160 anchors the retractor to the body after insertion of the wedge-shaped feature of the safety barrier shim into the ALL, between the ALL and the anterior face of a vertebral body, or between the ALL and the anterior of the disc space. Therefore, the safety barrier shim 1160 enables the retractor to secure itself to the anatomy of the body in the anterior portion of the disc space. The present inventor has recognized particular design advantages of the preferred embodiment of the safety barrier shim 1160, namely that the safety barrier shim 1160 in the present invention provides a leverage point to direct force from the anterior of the disc space to the posterior, in addition to providing a protective shield between the working channel and the structures anterior to the properly placed safety barrier shim 1160. In alternative embodiments of the invention, the stationary retractor blade 1110 itself may form the anchoring apparatus to provide a leverage point to direct force from the anterior of the disc space to the posterior, and thus provide a safety barrier between the working channel and anterior structures.

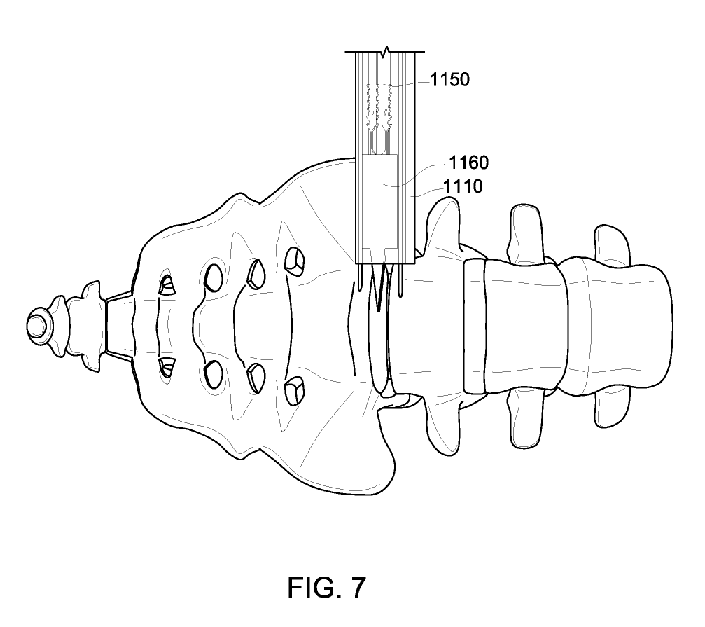

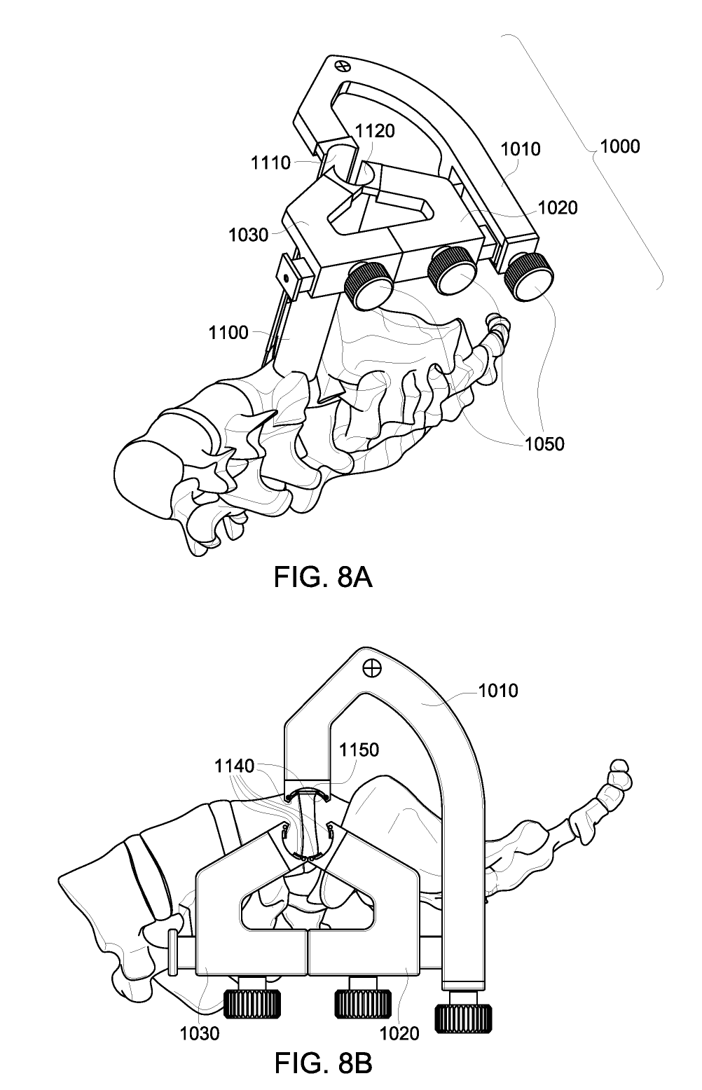

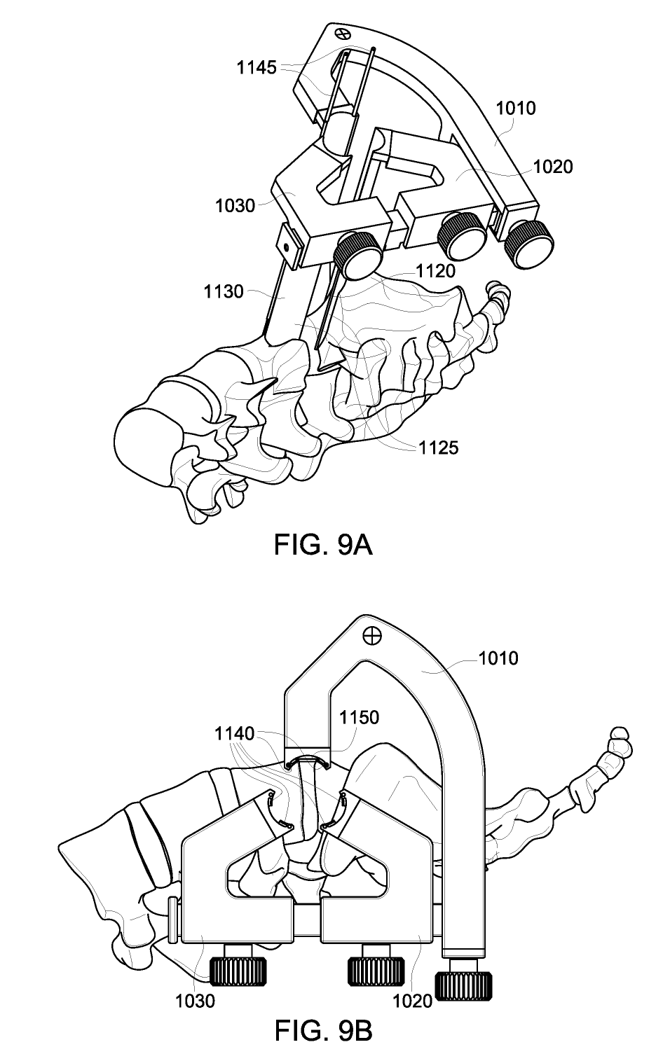

The preferred embodiment of the stationary retractor blade 1110 incorporates two or more pin channels 1140, configured to accommodate one or more locking pins 1145 as depicted in FIG. 9A and FIG. 9B. In an embodiment of the invention, the one or more locking pins 1145 are comprised of stainless steel. In an embodiment of the invention, the one or more locking pins 1145 have a diameter of 2.5 millimeters. In varying embodiments of the invention, the one or more locking pins 1145 have a diameter of 1 millimeter-4.5 millimeters. The pin channels 1140 are configured to allow placement of one or more locking pins 1145 through the stationary retractor blade 1110 in the preferred embodiment into one or more vertebral bodies adjacent to the disc space at the distal end of the working channel formed by the multi-bladed retractor assembly 1000. In embodiments, the movable retractor blades 1125 incorporate pin channels 1140, as depicted in FIG. 8B. In an embodiment of the invention, the diameter of the pin channel 1140 is 3 millimeters. In alternative embodiments of the invention, the diameter of the pin channels 1140 is any distance between 1 millimeter and 5 millimeters. In an embodiment, the pin channel may accommodate a neuromonitoring probe of a standard size as known to those skilled in the art. The present inventors have recognized the advantage of an embodiment of the present invention that the configuration of the multi-bladed retractor assembly as described herein allows for the operation of neuromonitoring probes during the process of opening the retractor blades 1100 from a closed to an open position, which represents a departure from other retractors known in the prior art. Moreover, the present inventors have recognized the advantage of an embodiment of the present invention that the configuration of the multi-bladed retractor assembly as described herein allows for the incorporation of a neuromonitoring probe into the pin channel 1140 in such a manner that allows a neuromonitoring probe to function during the opening of the movable retractor blades 1125 in a posterior trajectory. In alternative embodiments, the movable retractor blades 1125 incorporate blade slots to accommodate safety barrier shim 1160. As such, the locking pins 1145 when placed through the pin channels 1140 incorporated within the stationary retractor blade 1110 provide a mechanism for anchoring the multi-bladed retractor assembly 1000 into the patient's anatomy. In varying embodiments of the invention, the locking pins 1145 comprise a pin. In varying embodiments of the invention, the locking pins 1145 comprise a threaded screw. The diameter of the pin/screw fixation device in the preferred embodiment is 1.5-2 mm. In the preferred method of use, the surgeon may tap or screw each locking pin 1145 through a pin channel 1140 into a vertebral body at any depth up to 25 millimeters into the vertebral body.

In the varying methods of use associated with the invention, the user of the multi-bladed retractor assembly 1000 takes steps to secure it to patient anatomy. One such step is sliding the safety barrier shim 1160 along the slot of the stationary retractor blade 1110 into the anatomy near the anterior portion of the disc space, optionally into or abutting the ALL. Following the placement of the safety barrier shim 1160, another step to secure the multi-bladed retractor assembly 1000 to the patient anatomy is that of placing a locking pin 1145 through a pin channel 1140 in the stationary retractor blade 1110 into the bone of at least one vertebral body immediately cephalad or immediately caudal to a disc space. In the preferred method of use, the step of placing a pin/screw fixation device occurs repeatedly, by placing pin/screw fixation devices, which may include one or more locking pins 1145, through a pin channel 1140 in one or more blades of the multi-bladed retractor assembly 1000 into both of the vertebral bodies immediately cephalad and caudal to the disc space during a procedure. The present inventors also recognize that the patient-specific anatomy may prevent the placement of locking pins 1145 into both vertebral bodies adjacent to a disc space, including in scenarios involving the presence of osteophytes or scenarios in which a surgeon must alter the trajectory of the approach due to the presence of the iliac crest proximal to the desired approach path. Thus, prior to placing a pin/screw fixation device, the user of the multi-bladed retractor assembly 1000 engages in the step of evaluating the anatomy to determine appropriate placement of one or more pin/screw fixation devices. During this evaluating step, the user may consider the appropriate location of placement of one or more pin/screw fixation devices, and the appropriate number of pin/screw fixation devices to be placed considering the patient anatomy. In situations where the patient-specific anatomy prevents the placement of pin/screw fixation devices into both vertebral bodies adjacent to a disc space, the appropriate step therefore is placing a pin/screw fixation device into at least one of the vertebral bodies adjacent to a disc space.

After placement of the one or more pin/screw fixation devices, embodiments of the invention are associated with method steps intended to open a working channel. A step to accomplish the creation of a working channel is removing the one or more dilators and guide wire (also known as “Kirschner Wire” or “K-wire”). After removal of the dilators and guide wire, a user can then engage in the step of moving the retractor blades other than the stationary retractor blades. The movements associated with this step in embodiments of the invention involve moving the one or more movable retractor blades from a generally more anterior position to a generally more posterior direction, pushing the soft tissue substantially posterior to the one or more movable retractor blades in a generally posterior direction. More specifically, as the stationary retractor blade remains in a fixed position in or near what one skilled in the art recognizes as zones 1 and 2 of the disc space (the anterior portion), the one or more movable retractor blades move in a posterior direction toward and optionally into what one skilled in the art recognizes as zones 3 and 4 of the disc space (the posterior portion). Another optional step associated with opening the working channel is moving the movable retractor blades in a generally cephalad or caudal direction. In preferred embodiment of the invention, the multi-bladed retractor assembly 1000 is configured to have two movable retractor blades 1125, which may move independently from one another. Also in the preferred embodiment, the two movable retractor blades 1125, in addition to moving in a generally posterior direction from the stationary retractor blade, may move in a cephalad or caudal direction. In an embodiment of the invention, each of the stationary retractor blades 1110 and the movable retractor blades 1125 incorporate a toeing actuator to allow a user to pivot the blade around a toeing hinge 1040 located at or near the intersection of each retractor blade to its respective retractor arm. In an embodiment of the invention, the toeing actuation is accomplished via a toeing actuator 1060 as depicted in FIG. 16. In an embodiment of the invention, the toeing actuator 1060 comprises a set screw communicatively coupled to a worm gear which facilitates pivoting of a retractor blade around a toeing hinge 1040. In an embodiment of the invention, each blade incorporates a toeing actuator 1060. In an embodiment of the invention, each retractor arm incorporates a toeing actuator 1060. The present inventors recognize that a novel advantage of an embodiment of the present invention is the ability to independently control the toeing of each retractor blade toeing actuator 1060, which provides a high level of precision to surgeons, empowering them to control the dimensions of the working channel with a high level of fidelity.

The present inventors have recognized an advantage of the preferred embodiment of the invention, namely that it provides the ability to take the step of moving the movable retractor blades 1125 in a cephalad or caudal direction, in addition to the ability to take the step of moving the movable retractor blades 1125 in a generally posterior direction, which results in a larger opening that can accommodate larger-sized implants and instrumentation, and can provide a surgeon with a better range of visualization options for the surgeon through the working channel.

The present inventors have also recognized the advantage associated with the preferred embodiment of the invention, that the movements of the movable retractor blades 1125 both in the generally posterior direction and in the cephalad or caudal directions have a less traumatic effect upon the sensory nerves associated with zones 1 & 2 of the disc space. Movement of a movable retractor blade 1125 in such directions impacts the anatomy of the sensory nerves such that they specifically avoid elongation of the sensory nerves along or near the surgical approach path. This derives from the recognition of the atraumatic effects of moving the sensory nerves in a posterior direction from zones 1 & 2 of the disc space into zones 3 & 4. Moreover, the present inventors have recognized that as the sensory nerves are generally smaller in size than the motor nerves, by approaching the anterior third of the disc space, there is less of a risk of inadvertent contact with the nerves in the anterior portion of the disc space (the sensory nerves) than with the nerves of the posterior portions of the disc space (the motor nerves), representing another advantage to approaching the anterior aspects of the disc space.

The movement of the blades from a generally anterior position to a generally posterior position in association with embodiments of the present invention mitigates risk to the genito-femoral nerve (or “GFN”) specifically. In an embodiment of the invention, the placement of the stationary retractor blade 1110 in the anterior aspect of the disc space ensures that the GFN will either be anterior to the stationary retractor blade 1110 after placement and therefore untouched, or that the GFN will be retracted from zones 1 and 2 of the disc space (the anterior portion) into zones 3 and 4 of the disc space (the posterior portion) in an atraumatic fashion. In association with an intended method of use in an embodiment of the invention, the lordotic curve of the GFN relative to the direction of retraction ensures that the risk of indirect damage to the GFN due to elongation is minimized as a result. The concern associated with the difficulty in detecting the GFN as a sensory nerve is mitigated by embodiments of the present invention. The preferred embodiment of the invention therefore solves a primary concern of surgeons when considering an approach to the anterior third of the disc space, namely, damage to the GFN. Embodiments of the invention take account of the smaller and more pliable nature of the sensory nerves, and their increased resilience to non-elongating movements relative to the motor nerves.

The techniques and apparatuses associated with accomplishing the moving step do so specifically by utilizing apparatuses such as conically-shaped dilators 1400 incorporating atraumatic shapes to avoid elongation or direct damage to the GFN. The dilators 1400 associated with embodiments of the invention incorporate an atraumatic distal end with a rounded and more gentle tip. The tip of the preferred embodiment of the dilator 1400 incorporates a substantially rounded or blunt, atraumatic tip having a radius of no less than 4 mm. The distal end of the dilators 1400 associated with embodiments of the invention also incorporate a rounded cut, as opposed to a straight cut, thereby mitigating damage to nerves caused by the placement of the distal end of the dilator 1400. The method steps associated with embodiments of the invention specifically approaching the area containing the GFN with techniques and apparatuses designed to move it in a direction that will avoid elongation, thereby minimizing risk of damage to the GFN, thereby solving a previously unmet need.

Additionally, the present inventors have recognized that movements of the retractor blades 1100 both in the generally posterior direction and in the cephalad or caudal directions have a less traumatic effect upon the musculature, especially the psoas muscle, located near the anterior portion of the disc space. This stems from the fact that less musculature generally exists in and near zones 1 and 2 of the disc space relative to the musculature that exists in and near zones 3 and 4 of the disc space. By approaching the anterior aspect of the disc space, therefore, the surgeon is less likely to incur direct or indirect musculature damage because of the lower presence of musculature in that area.

### PREFERRED METHOD OF USE

Preferred method steps associated with embodiments of the invention:
a. In the preferred embodiment of the invention, the method steps associated with the approach progress comprise the following:

Targeting the incision in the skin over the anterior third of the disc space;
a. Adjusting fluoroscopy positioning and patient positioning so that the imaging of the endplates are clearly identifiable on fluoroscopy imaging in the true lateral place and in the anterior-posterior plane;
b. Laying an elongate member 1410, such as a Kirchner Wire or guide wire, across the exterior skin to create a reference point;
c. Identifying and locating the anterior, posterior, superior, and inferior aspect of the targeted disc space;
d. Marking the previously identified anterior, posterior, superior and inferior aspects of the targeted disc space on the skin of a patient;
e. Placing an incision on the intended trajectory from the skin to the targeted anterior third of the disc space;

Dissecting to the top of the psoas muscle with the finger;

Sequentially dilating to the anterior third of the interbody disc;
a. Seeking the posterior aspect of the anterior longitudinal ligament to identify the anterior aspect of the disc space by continuously reviewing fluoroscopy;
b. Placing an atraumatic cannulated dilator through the soft tissue between the skin and the spine to the point where the distal end of the atraumatic cannulated dilator comes into contact with the anterior aspect of the disc space generally equidistant from the adjacent superior vertebral body and the adjacent inferior vertebral body (the “anterior disc target location”);
c. Optionally determining neural proximity by utilization of a neuro-monitoring mechanism coupled with one or more dilators 1400;
d. Checking fluoroscopic imaging in both the lateral plane and the anterior-posterior plane to assure proper placement of the distal end of the dilator in contact with the anterior disc target location;
e. Sliding guide wire through the cannulation of the atraumatic cannulated dilator and into the disc space;
   i. Advancing the guide wire through the cannulation of the atraumatic cannulated dilator;
   ii. Piercing the annulus of the disc space;
   iii. Placing the distal tip of the guide wire approximately half way through the disc;
f. Optionally expansively dilating to the targeted disc space by using an oval shaped dilator 1430;
   i. Placing an oval shaped dilator 1430 over the atraumatic cannulated dilator and guide wire, with its major axis aligned in a substantially cephalad/caudal orientation in line with the psoas muscle fibers;
   ii. Sliding the oval shaped dilator 1430 through the oblique and psoas tissues until the distal end of the oval shaped dilator 1430 is fully in contact with the disc space and length-wise even with distal end of the atraumatic cannulated dilator;
   iii. Optionally determining neural proximity by utilization of a neuro-monitoring mechanism coupled with the oval shaped dilator 1430;
   iv. Rotating the oval shaped dilator 1430 approximately 90 degrees, with its major axis now aligned in a substantially anterior/posterior orientation, to displace tissue adjacent to the anterior aspect of the disc space and near the intended working channel, thereby creating a pathway for the retractor;
   v. Optionally determining neural proximity by utilization of a neuro-monitoring mechanism coupled with one or more dilators 1400;
g. Optionally dilating to the targeted disc space by using a second round shaped dilator;
   i. Sliding a second round shaped dilator over the atraumatic cannulated dilator and guide wire;
   ii. Advancing the second round shaped dilator through the oblique and psoas tissues until its distal end is in contact with the anterior aspect of disc space, optimally the anterior disc target location, and substantially even with the distal end of the atraumatic cannulated dilator and the distal end of the guide wire;
   iii. Optionally determining neural proximity by utilization of a neuro-monitoring mechanism coupled with one or more dilators;
   iv. Sliding a third dilator over the second round shaped dilator;
   v. Advancing the third dilator through the oblique and psoas tissue until the distal end is in contact with the anterior aspect of disc space or anterior disc target location and substantially even with the distal end of the second dilator, the distal end of the atraumatic cannulated dilator and the distal end of the guide wire;
   vi. Optionally determining neural proximity by utilization of a neuro-monitoring mechanism coupled with one or more retractor blades;
h. Enveloping the proximal end of the outermost dilator with the distal end of the retractor blades in compressed form;
i. Advancing the retractor blades 1100 slidably along the outermost dilator such that the distal end of the stationary dilator blade 1110 is positioned in the anterior aspect of the disc space, optimally the anterior disc target location, at a point generally posterior to the anterior longitudinal ligament (ALL);
j. Optionally determining neural proximity by utilization of a neuro-monitoring mechanism coupled with one or more retractor blades;
k. Coupling a table mounted retractor arm (TMRA), in a loose configuration, to the retractor;
l. Confirming proper placement of the distal end of the multi-bladed retractor assembly 1000 in contact with the anterior aspect of the disc, optimally the anterior disc target location, by checking fluoroscopic imaging in the lateral plane and fluoroscopic imaging in the anterior-posterior plane;
m. Tightening the TMRA to lock it in the fixed position;
n. Placing the safety barrier shim 1160 into a groove of the stationary retractor blade 1110;
o. Pressing the safety barrier shim 1160 into a secure position into the anterior aspect of the disc space immediately posterior to the ALL to secure the multi-bladed retractor assembly 1000 and create a protective barrier;
p. Evaluating the patient anatomy for locking pin 1145 placement;
q. Securing one or two more locking pins 1145 coupled with one or more retractor blades 1100 to one or more of the superior and inferior vertebral bodies;
r. Removing the one or more dilators 1400 and the guide wire;
s. Expanding one or more of the movable blades 1125 of the multi-bladed retractor assembly 1000 in a generally posterior direction by actuating a linear motion that independently moves the proximal blade 1120 or the distal blade 1130, or both the proximal blade 1120 and distal blade 1130 together, or the single movable blade in a two blade configuration, in a generally posterior direction using mechanically guided motion from the surgeon, or a ratchet and pawl mechanism, a worm gear mechanism or a drive screw mechanism integrated into the multi-bladed retractor assembly 1000;
t. Optionally, turning the toeing actuator 1060 to create towing of the blade such that the distal end of the blade moves in an outward direction from the dilation axis rotating around the towing hinge 1040.
u. Locking into position once the desired retraction is achieved with a spring lock or lock nut as a part of the drive mechanism, thereby creating a sufficient working channel;
v. Optionally determining neural proximity by utilization of a neuro-monitoring mechanism coupled with one or more of the retractor blades 1100 via one or more pin channels 1140;
w. Optionally independently expanding a proximal blade 1120 or distal blade 1130 in its respective cephalad or caudal direction independently of the other movable retractor blade;
x. Optionally repeating the independent movements of one or a proximal blade 1120 or distal blade 1130 until the surgeon determines that the opening of the working channel is of sufficient dimensions;
y. Optionally determining neural proximity by utilization of a neuro-monitoring mechanism coupled with one or more retractor blades;
z. Confirming proper placement of the distal end of the retractor in contact with the anterior aspect of the disc by checking fluoroscopic imaging in the lateral plane and fluoroscopic imaging in the anterior-posterior plane;

After these steps, performing the functions associated with discectomy, interbody placement and instrument removal as known in the prior art, including collapsing and removing the retractor.

In the foregoing specification, specific embodiments have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings.

The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

Moreover in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art. The terms “coupled” and “linked” as used herein is defined as connected, although not necessarily directly and not necessarily mechanically. A device or structure that is “configured” in a certain way is configured in at least that way, but may also be configured in ways that are not listed. Also, the sequence of steps, whether described in the text of the specification, in a flow diagram or elements in the claims, even when preceded by a number or letter does not imply or require that sequence.

1. A system for forming an operating corridor to a lumbar spine, comprising:
an elongate member deliverable to a spinal disc along a lateral path to the lumbar spine;
a dilator system to create a distraction corridor along the lateral path to the lumbar spine, the dilator system comprising at least one dilator cannula that slidably engages an exterior of the elongate member, the at least one dilator being deliverable to the spinal disc along the lateral path to the lumbar spine;
a multi-bladed retractor assembly slidable over the dilator system toward the spinal disc along the lateral path, the multi-bladed retractor assembly including:
a curved stationary arm engaging a stationary retractor blade that extends generally perpendicularly relative to the curved stationary arm and at least one movable retractor arm engaging a movable retractor blade that extends generally perpendicularly relative to the movable retractor arm,
wherein the multi-bladed retractor assembly is adjustable from a closed position in which the stationary and at least one movable retractor blade are adjacent to one another and slidable over the dilator system to an opened position in which the at least one movable retractor blade is moved away from the stationary retractor blade to enlarge the distraction corridor and thereby form an operative corridor along the lateral to the lumbar spine,
wherein the stationary retractor blade is deliverable to the anterior aspect of the spinal disc,
wherein the at least one movable retractor blade is initially adjustable from a closed position in a posterior trajectory relative to the stationary retractor blade delivered to the anterior aspect of the spinal disc, and
wherein when the multi-bladed retractor assembly is adjusted to the opened position to form the operative corridor along the lateral path to the lumbar spine, such that the at least one movable retractor blade is positioned to open the operative corridor to a dimension so as to pass an implant through the operative corridor and into the lumbar spine.

2. The system of claim 1, wherein the simultaneous movement of the one or more movable retractor blades orthogonally relative to the stationary blade occurs in response to rotation of an end knob.

3. The system of claim 1, wherein each of the one or more movable blades are independently movable along the caudal-cephalad plane.

4. The system of claim 3, wherein the independent movement of each of the movable retractor blades in a caudal-cranial plane blade occurs in response to rotation of a movement actuator.

5. The system of claim 1, comprising retractor blades that pivot around a toeing hinge in response to rotation of a toeing actuator.

6. The system of claim 1, wherein a safety barrier shim is slidable into a position in the anterior aspect of the disc via a sliding movement along a blade slot in the stationary retractor blade.

7. The system of claim 1, wherein a safety barrier shim is slidable to a position in the anterior aspect of a disc space via a sliding movement along a blade slot in the stationary retractor blade.

8. The system of claim 1, wherein at least one retractor blade incorporates a pin channel.

9. The system of claim 1, wherein the stationary retractor blade and the at least one movable retractor blade incorporate a pin channel dimensioned to accommodate a neuromonitoring probe operable during the process of adjustment of the at least one movable retractor blade from its closed position to its opened position.

10. The system of claim 1, wherein the cross-sectional shape of at least one of the dilators comprises an oval.

11. The system of claim 1, comprising at least one toeing hinge.

12. The system of claim 1, comprising a final dilator, wherein the shape of the cross-section of the final dilator is hexagonal.

13. The system of claim 1, wherein the stationary blade is positionable with its external face away from a user.

14. The system of claim 1, wherein the multi-bladed retractor assembly is capable of transitioning from an anterior to posterior expansion position to a traditional posterior to anterior retraction expansion position by reversing the orientation of the multi-bladed retractor assembly.

15. The system of claim 1, wherein the multi-bladed retractor assembly comprises one stationary retractor blade, one movable blade proximal to a stationary retractor arm and another movable retractor blade on the opposite side of the movable blade proximal to the stationary retractor arm.

16. The system of claim 1, wherein at least one dilator exhibits the cross-sectional shape of an oval and wherein the blades of the multi-bladed retractor assembly in the closed position correspondingly exhibit the cross-sectional shape of a slightly larger oval.

17. A method for forming an operating corridor to a lumbar spine, comprising:
targeting an incision point on the skin of a patient to open a pathway to an anterior third of a disc space;
marking the incision point;
incising the skin at the incision point;
dilating to the anterior third of the disc space by placement of a series of dilators;
enveloping a proximal end of an outer-most dilator with a distal end of a plurality of retractor blades of a multi-bladed retractor assembly;
advancing the plurality of retractor blades of the multi-bladed retractor assembly along the outer-most dilator to the anterior aspect of the disc space;
removing the series of dilators;
expanding the retractor blades in a generally posterior direction.

18. The method of claim 17, further comprising:
placing a safety barrier shim into a blade slot of a retractor blade;
pressing the safety barrier shim to a point within or just anterior to the anterior aspect of the disc space.

19. The method of claim 17 further comprising:
coupling a table-mounted retractable arm to the multi-bladed retractor assembly;
locking the table-mounted retractable arm into position.

20. The method of claim 17, further comprising:
evaluating the patient anatomy for locking pin placement;
securing a locking pin coupled with a retractor blade to an adjacent vertebral body.

21. The method of claim 17, further comprising:
locking the retractor blades into position when a desired level of retraction is achieved.

22. The method of claim 17, wherein the dilator used during the dilating step comprises an oval shaped dilator, and a major axis of the oval shaped dilator is aligned substantially with the cephalad/caudal orientation and the psoas muscle fibers,
wherein dilating step further comprises rotating the oval shaped dilator 90-degrees,
wherein the major axis of the oval shaped dilator aligns in a substantially anterior/posterior orientation.

23. The method of claim 17, wherein the dilator of the dilating step comprises a round shaped dilator;
and the dilating step further comprising sliding a third dilator over a second dilator through the oblique and psoas muscles until the distal end of the third dilator is in contact with the anterior aspect of the disc space.

24. The method of claim 17, further comprising the step of neuromonitoring to determine neuroproximity of a dilator.

25. The method of claim 17, further comprising the step of securing a plurality of fixation devices coupled with the retractor blades to adjacent vertebral bodies in the cephadal and caudal directions.
